SEQUENCE LISTING (6 sequences)

SEQ ID NO: 1 - DNA, 20 bp, Artificial Sequence
  primer
ctcgttcgctcgcctggcgc

SEQ ID NO: 2 - DNA, 20 bp, Artificial Sequence
  primer
gaagttgcgcatcatgctgt

SEQ ID NO: 3 - DNA, 20 bp, Artificial Sequence
  primer
tccatccagcagaccacgct

SEQ ID NO: 4 - RNA, 19 nt, Artificial Sequence
  primer
gccggacaccacacggaag

SEQ ID NO: 5 - DNA, 20 bp, Artificial Sequence
  primer
atgcgcttctccagctttgt

SEQ ID NO: 6 - DNA, 20 bp, Artificial Sequence
  primer
tatggagcacacctcacctg

(19) United States
(12) Patent Application Publication
(10) Pub. No.: US 2007/0009943 A1
(43) Pub. Date: Jan. 11, 2007

(54) Methods, uses and compositions for determining redisposition to cancers by identifying specific genotypes of CYP1B1 gene

(76) Inventors: Jan Lubinski, Szecin (PL); Joanna Matyjasik, Police (PL)

Correspondence Address: COOPER & DUNHAM, LLP, 1185 AVENUE OF THE AMERICAS, NEW YORK, NY, 10036, US

(21) Appl. No.: 11/474,160

(22) Filed: Jun. 23, 2006

Related U.S. Application Data
(60) Provisional application No. 60/693,149, filed on Jun. 23, 2005.

(51) Int. Cl. C12Q 1/68; C12P 19/34

(52) U.S. Cl. 435/6; 435/91.2

(57) ABSTRACT

The present invention is provides the methods, uses and compositions useful in diagnosis of low-penetrance predisposition to human cancers of various sites wherein constitutional variants of alterations within CYP1B1 gene are analyzed in biological material from examined individual.

This application claims benefit of U.S. Provisional application No. 60/693,149, filed Jun. 23, 2005, the contents of which are hereby incorporated herein by reference.

Documents are cited throughout the text of this specification. Each of the documents cited herein (including any manufacturer's specifications, instructions, etc.) are hereby incorporated herein by reference; however, there is no admission that any document cited is indeed prior art as to the present invention.

### FIELD OF THE INVENTION

Mode and composition for detection of inherited predisposition to cancers of various sites and the use of germline variants within CYP1B1 gene for diagnosing of such predisposition are subject of invention. Generally, the invention concerns the new diagnostic method. Subject of invention allow to synthesize DNA and identification of genomic abnormalities which are correlated with increased/decreased genetic predisposition to cancers of various organs.

### BACKGROUND OF THE INVENTION

CYP1A1 and CYP1B1 are two major enzymes of cytochrome P450 involved in carcinogenic hydroxylation [10, 16,17]. CYP1B1 exceeds CYP1A1 in its catalytic efficiency as a hydroxylase [14]. This indicates that CYP1B1 is one of key players in the carcinogenesis process.

The hydroxylation activity of CYP1B1 is of particular importance, since activated carcinogens induce DNA single-strand breakages [8] and mutations [3]. The cytochrome P450 1B1 metabolises estradiol to 4-hydroxyestradiol, which is a catechol estrogen that has been shown to induce depurination of DNA that leads to DNA mutations [1,3,18]. In addition to estrogens, CYP1B1 also bioactivates a range of chemically diverse procarcinogens including benzo[a]pyrene [12,14,20]. Sequences of naturally-ocurring human CYP1B1 nucleic acid are described in GenBank™ Accession (GB#U56438) and Tang et al. (1996) J. Biol. Chem. 271:28324.

Several studies indicate that some variants of CYP1B1 are showing much stronger carcinogenic hydroxylation activities [9,14,15]. The potential relationship between various single nucleotide polymorphism sites (SNPs) present in the human CYP1B1 gene and the incidence of different cancer types has been suggested [5,6,20].

Several polymorphisms of the CYP1B1 gene have been described. Four of them—at codon 48, 119, 432 and 453, results in amino acid replacement. Additionally, polymorphisms at codon 119 and 432 are showing high catalytic activity in region with recognized functional activity—heme-binding region and presumed substrate recognition site 1 (SRS1) of CYP1B1 [7,20].

International patent application WO 02/04683 discloses the mode of identification of persons with increased risk of estrogen-dependent cancers, particularly cancers of the breast, wherein CYP1B1, CYP1A1, COMT and GSTM1 genotypes are examined. Among SNPs occurring on CYP1B1 gene only codons 432 and 453 are disclosed as significant in WO 02/04683. U.S. Patent application publication US 20040002071 discloses the mode of assessment of breast cancer risk by examination of at least two genes. In CYP1B1, US 20040002071 identifies two CYP1B1 alleles containing G or C at nucleotide position 1294 (Gene Bank accession No U 56438) which corresponds to codon 432, as alleles indicating an increased probability of cancer.

Up to recently, any definite conclusion could not be taken from cancer association studies on 355 T/T variant. Japanese groups reported increased risk of cancers of the prostate and kidney among carriers of 355T/T variant [13,19]. However both of studies were performed on unacceptably short series of individuals (117 and 211 of cases respectively; 200 controls).Studies on Swedish women did not show association between 355 T/T and cancer risk, at least for postmenopausal women, of breast and endometrial cancers, although the latest studies were performed on large groups—1521 breast cancers, 689 endometrial cancers and around 1500 controls [12].

Despite of longstanding research efforts aimed to develop a mode of diagnosing increased or decreased predisposition to cancers of various sites, such diagnostic methods are still needed This invention is aimed to provide modes and compositions which can be useful in detection of increased/decreased inherited predisposition to cancers of various sites.

### SUMMARY OF THE INVENTION

The subject of invention provides a method for detection of genetic predisposition to cancers at various sites. The method comprises analysis of biological material obtained from the patient and searching for homozygous 355T/T or combined genotypes based on C142G, G355T and C4326G SNPs within CYP1B1 gene; the presence of any of the variants presented in Table A and B in the sample from the patient indicates significantly increased (Table A) or decreased (Table B) predisposition to cancers at the following sites: breast, colon, kidney, larynx, lung, pancreas, prostate and, with high probability, thyroid and ovarian.

### DETAILED DESCRIPTION

Disclosed is a method for determining predisposition of a human subject to cancer comprising determining whether the CYP1B1 gene of the human subject has the genotype 355 T/T, 355 G/T, 355 G/G, 142 G/C, 142 C/C, 142 G/G, 4329 C/C, 4329 G/C or 4329 G/C, wherein the presence of the genotype is indicative of predisposition of the human subject to cancer.

In an embodiment of the method, determining predisposition of a human subject to cancer comprises determining whether the CYP1B1 gene of the human subject has at least two codon variations selected from the group consisting of A119S alteration, R48G alteration and L432V alteration, wherein the presence of the codon variations is indicative of predisposition of the human subject to cancer. In another embodiment, screening is performed for all three codon variations.

In the method, the A119S alteration results from the genotype 355 T/T, 355 G/T or 355 G/G, the R48G alteration results from the genotype 142 G/C, 142 C/C or 142 G/G; and the L432V alteration results from the genotype 4329 C/C, 4329 G/C or 4329 G/G. Specific genotype of CYP1B1 gene indicative of significantly increased predisposition to a specific cancer or group of cancers is as follows:
CYP1B1 gene combined genotype 4329 C/C, 142 G/G, 355 T/T being indicative of significantly increased predisposition to at least one of the following cancers: cancers of the breast, colon, larynx, lung, pancreas or prostate,
CYP1B1 gene combined genotype 4329 C/C, 142 C/C, 355 G/T being indicative of significantly increased predisposition to at least one of the following cancers: cancers of the breast or colon,
CYP1B1 gene combined genotype 4329 C/C, 142 G/C, 355 T/T being indicative of significantly increased predisposition to larynx cancer,
CYP1B1 gene combined genotype 4329 C/C, 142 G/G, 355 G/G being indicative of significantly increased predisposition to colon cancer,
CYP1B1 gene combined genotype 4329 G/C, 142 G/G, 355 G/T being indicative of significantly increased predisposition to prostate cancer,
CYP1B1 gene combined genotype 4329 G/C, 142 C/C, 355 G/G being indicative of significantly increased predisposition to breast cancer,
CYP1B1 gene combined genotype 4329 G/C, 142 C/C, 355 G/T being indicative of significantly increased predisposition to at least one of the following cancers: cancers of the breast or colon,
CYP1B1 gene combined genotype 4329 G/G, 142 C/C, 355 G/G being indicative of significantly increased predisposition to kidney cancer,
CYP1B1 gene combined genotype 4329 C/C, 355 T/T being indicative of significantly increased predisposition to at least one of the following cancers: cancers of the breast, larynx, pancreas or prostate,
CYP1B1 gene combined genotype 4329 C/C, 355 G/T being indicative of significantly increased predisposition to lung cancer,
CYP1B1 gene combined genotype 4329 G/G, 355 G/G being indicative of significantly increased predisposition to kidney cancer,
CYP1B1 gene combined genotype 4329 G/G, 355 T/T being indicative of significantly increased predisposition to colon cancer,
CYP1B1 gene combined genotype 4329 C/C, 142 G/G being indicative of significantly increased predisposition to at least one of the following cancers: cancers of the breast or prostate,
CYP1B1 gene combined genotype 4329 C/C, 142 G/C being indicative of significantly increased predisposition to larynx cancer,
CYP1B1 gene combined genotype 4329 G/C, 142 C/C being indicative of significantly increased predisposition to breast cancer,
CYP1B1 gene combined genotype 4329 G/G, 142 C/C being indicative of significantly increased predisposition to kidney cancer,
CYP1B1 gene combined genotype 142 C/C, 355 G/G being indicative of significantly increased predisposition to at least one of the following cancers: cancers of the breast, kidney or lung,
CYP1B1 gene combined genotype 142 C/C, 355 G/T being indicative of significantly increased predisposition to at least one of the following cancers: cancers of the breast or colon,
CYP1B1 gene combined genotype 142 C/C, 355 T/T being indicative of significantly increased predisposition to colon cancer,
CYP1B1 gene combined genotype 142 G/C, 355 T/T being indicative of significantly increased predisposition to larynx cancer,
CYP1B1 gene combined genotype 142 G/G, 355 G/G being indicative of significantly increased predisposition to colon cancer,
CYP1B1 gene combined genotype 142 G/G, 355 T/T being indicative of significantly increased predisposition to at least one of the following cancers: cancers of the breast or prostate.

Specific genotype of CYP1B1 gene indicative of significantly decreased predisposition to a specific cancer or group of cancers is as follows:
CYP1B1 gene combined genotype 4329 C/C, 142 G/G, 355 G/G being indicative of significantly decreased predisposition to breast cancer,
CYP1B1 gene combined genotype 4329 C/C, 142 G/C, 355 G/G being indicative of significantly decreased predisposition to at least one of the following cancers: cancers of the breast or prostate,
CYP1B1 gene combined genotype 4329 C/C, 142 G/G, 355 G/T being indicative of significantly decreased predisposition to breast cancer,
CYP1B1 gene combined genotype 4329 G/C, 142 G/C, 355 G/G being indicative of significantly decreased predisposition to at least one of the following cancers: breast, larynx, lung or prostate,
CYP1B1 gene combined genotype 4329 G/G, 142 C/C, 355 G/T being indicative of significantly decreased predisposition to breast cancer,
CYP1B1 gene combined genotype 4329 G/G, 142 G/C, 355 G/T being indicative of significantly decreased predisposition to at least one of the following cancers: cancers of the breast, lung or prostate,
CYP1B1 gene combined genotype 4329 G/G, 355 T/T being indicative of significantly decreased predisposition to breast cancer,
CYP1B1 gene combined genotype 4329 G/G, 355 G/T being indicative of significantly decreased predisposition to at least one of the following cancers: cancers of the breast, larynx, lung, pancreas or prostate,
CYP1B1 gene combined genotype 4329 G/C, 355 T/T being indicative of significantly decreased predisposition to breast cancer,
CYP1B1 gene combined genotype 4329 G/C, 142 G/G being indicative of significantly decreased predisposition to colon cancer,
CYP1B1 gene combined genotype 4329 G/G, 142 G/C being indicative of significantly decreased predisposition to at least one of the following cancers: cancers of the breast, larynx, lung or pancreas,
CYP1B1 gene combined genotype 4329 G/C, 142 G/C being indicative of significantly decreased predisposition to at least one of the following cancers: cancers of the breast or prostate,
CYP1B1 gene combined genotype 142 C/C, 355 G/T being indicative of significantly decreased predisposition to lung cancer,
CYP1B1 gene combined genotype 142 G/C, 355 G/G being indicative of significantly decreased predisposition to at least one of the following cancers: cancers of the breast, larynx, lung or prostate,
CYP1B1 gene combined genotype 142 G/G, 355 G/G being indicative of significantly decreased predisposition to the prostate cancer,
CYP1B1 gene combined genotype 142 G/G, 355 G/T being indicative of significantly decreased predisposition to at least one of the following cancers: cancers of the breast or larynx.

Another embodiment of the invention is a method for detecting a predisposition to cancer in a human subject, comprising detecting in a biological sample from the subject a germline alteration in sequence of CYP1B1 gene and identification of the CYP1B1 genotype, wherein the genotype is indicative of predisposition to at least one of the following cancers: cancers of the breast, colon, kidney, larynx, lung, pancreas, prostate, thyroid and ovarian. The examined alteration in sequence of CYP1B1 gene may be at least one of the following alteration:
variants of A119S alteration: 355T/T, 355G/T or 355G/G,
variants of R48G alteration: 142G/C, 142C/C, 142G/G
variants of L432V alteration: 4329C/C, 4329G/C, 4329G/G
or other CYP1B1 alterations with analogous properties.

In the method, the identified genotype of CYP1B1 gene being indicative of significantly increased predisposition to cancer is at least one of the following combined genotypes:
CYP1B1 gene combined genotype 4329 C/C, 142 G/G, 355 T/T being indicative of significantly increased predisposition to at least one of the following cancers: cancers of the breast, colon, larynx, lung, pancreas or prostate,
CYP1B1 gene combined genotype 4329 C/C, 142 C/C, 355 G/T being indicative of significantly increased predisposition to at least one of the following cancers: cancers of the breast or colon,
CYP1B1 gene combined genotype 4329 C/C, 142 G/C, 355 T/T being indicative of significantly increased predisposition to larynx cancer,
CYP1B1 gene combined genotype 4329 C/C, 142 G/G, 355 G/G being indicative of significantly increased predisposition to colon cancer,
CYP1B1 gene combined genotype 4329 G/C, 142 G/G, 355 G/T being indicative of significantly increased predisposition to prostate cancer,
CYP1B1 gene combined genotype 4329 G/C, 142 C/C, 355 G/G being indicative of significantly increased predisposition to breast cancer,
CYP1B1 gene combined genotype 4329 G/C, 142 C/C, 355 G/T being indicative of significantly increased predisposition to at least one of the following cancers: cancers of the breast or colon,
CYP1B1 gene combined genotype 4329 G/G, 142 C/C, 355 G/G being indicative of significantly increased predisposition to kidney cancer,
CYP1B1 gene combined genotype 4329 C/C, 355 T/T being indicative of significantly increased predisposition to at least one of the following cancers: cancers of the breast, larynx, pancreas or prostate,
CYP1B1 gene combined genotype 4329 C/C, 355 G/T being indicative of significantly increased predisposition to lung cancer,
CYP1B1 gene combined genotype 4329 G/G, 355 G/G being indicative of significantly increased predisposition to kidney cancer,
CYP1B1 gene combined genotype 4329 G/G, 355 T/T being indicative of significantly increased predisposition to colon cancer,
CYP1B1 gene combined genotype 4329 C/C, 142 G/G being indicative of significantly increased predisposition to at least one of the following cancers: cancers of the breast or prostate,
CYP1B1 gene combined genotype 4329 C/C, 142 G/C being indicative of significantly increased predisposition to larynx cancer,
CYP1B1 gene combined genotype 4329 G/C, 142 C/C being indicative of significantly increased predisposition to breast cancer,
CYP1B1 gene combined genotype 4329 G/G, 142 C/C being indicative of significantly increased predisposition to kidney cancer,
CYP1B1 gene combined genotype 142 C/C, 355 G/G being indicative of significantly increased predisposition to at least one of the following cancers: cancers of the breast, kidney or lung,
CYP1B1 gene combined genotype 142 C/C, 355 G/T being indicative of significantly increased predisposition to at least one of the following cancers: cancers of the breast or colon,
CYP1B1 gene combined genotype 142 C/C, 355 T/T being indicative of significantly increased predisposition to colon cancer,
CYP1B1 gene combined genotype 142 G/C, 355 T/T being indicative of significantly increased predisposition to larynx cancer,
CYP1B1 gene combined genotype 142 G/G, 355 G/G being indicative of significantly increased predisposition to colon cancer,
CYP1B1 gene combined genotype 142 G/G, 355 T/T being indicative of significantly increased predisposition to at least one of the following cancers: cancers of the breast or prostate.

Also in the method, the identified genotype of CYP1B1 gene being indicative of significantly decreased predisposition to cancer is at least one of the following combined genotypes:
CYP1B1 gene combined genotype 4329 C/C, 142 G/G, 355 G/G being indicative of significantly decreased predisposition to breast cancer,
CYP1B1 gene combined genotype 4329 C/C, 142 G/C, 355 G/G being indicative of significantly decreased predisposition to at least one of the following cancers: cancers of the breast or prostate,
CYP1B1 gene combined genotype 4329 C/C, 142 G/G, 355 G/T being indicative of significantly decreased predisposition to breast cancer,
CYP1B1 gene combined genotype 4329 G/C, 142 G/C, 355 G/G being indicative of significantly decreased predisposition to at least one of the following cancers: breast, larynx, lung or prostate,
CYP1B1 gene combined genotype 4329 G/G, 142 C/C, 355 G/T being indicative of significantly decreased predisposition to breast cancer,
CYP1B1 gene combined genotype 4329 G/G, 142 G/C, 355 G/T being indicative of significantly decreased predisposition to at least one of the following cancers: cancers of the breast, lung or prostate,
CYP1B1 gene combined genotype 4329 G/G, 355 T/T being indicative of significantly decreased predisposition to breast cancer,
CYP1B1 gene combined genotype 4329 G/G, 355 G/T being indicative of significantly decreased predisposition to at least one of the following cancers: cancers of the breast, larynx, lung, pancreas or prostate,
CYP1B1 gene combined genotype 4329 G/C, 355 T/T being indicative of significantly decreased predisposition to breast cancer,
CYP1B1 gene combined genotype 4329 G/C, 142 G/G being indicative of significantly decreased predisposition to colon cancer,
CYP1B1 gene combined genotype 4329 G/G, 142 G/C being indicative of significantly decreased predisposition to at least one of the following cancers: cancers of the breast, larynx, lung or pancreas,
CYP1B1 gene combined genotype 4329 G/C, 142 G/C being indicative of significantly decreased predisposition to at least one of the following cancers: cancers of the breast or prostate,
CYP1B1 gene combined genotype 142 C/C, 355 G/T being indicative of significantly decreased predisposition to lung cancer,
CYP1B1 gene combined genotype 142 G/C, 355 G/G being indicative of significantly decreased predisposition to at least one of the following cancers: cancers of the breast, larynx, lung or prostate,
CYP1B1 gene combined genotype 142 G/G, 355 G/G being indicative of significantly decreased predisposition to the prostate cancer,
CYP1B1 gene combined genotype 142 G/G, 355 G/T being indicative of significantly decreased predisposition to at least one of the following cancers: cancers of the breast or larynx.

In the disclosed methods, the genetic testing is performed among all adults; the presence of germline alteration is detected by analysis of DNA, RNA or proteins, wherein DNA or RNA testing may be performed with the use of any technique of indirect mutation detection, more preferably selected among ASA-, ASO-, RFLP-PCR, microarrays or methods of direct mutation detection such as sequencing.

Also in the disclosed methods, the presence of the polypeptide encoded by CYP1B1 allele with germ line alteration is detected with the use of antibodies or other substances specific for this polypeptide or its fragment.

The investigated human subject may be a person of Polish ethnic origin.

Another embodiment of the invention is a diagnostic composition for detecting predisposition to cancer in a human subject, comprising at least two different oligonucleotides allowing amplification of region of genome of said human subject containing at least one of the following alteration:
variants of A119S alteration: 355T/T, 355G/T or 355G/G,
variants of R48G alteration: 142G/C, 142C/C, 142G/G
variants of L432V alteration: 4329C/C, 4329G/C, 4329G/G
or other alterations in linkage disequilibrium with above variants, wherein the detected predisposition to cancer is significantly increased/decreased predisposition to at least one of the following cancers: cancers of the breast, colon, kidney, larynx, lung, pancreas, prostate, thyroid and ovarian.

In the diagnostic composition, the amplified region is used for identification of the CYP1B1 genotype, wherein specific combined CYP1B1 genotypes defined herein in Table A are indicative of significantly increased predisposition to specific cancer, and specific combined CYP1B1 genotypes defined herein in Table B are indicative of significantly decreased predisposition to cancer.

Also provided by this invention is a method of identifying a genetic marker indicative of significantly increased/decreased predisposition to cancer, comprising examination of samples containing genomic DNA from patients affected by specific cancer and comparing the frequency of structural change within CYP1B1 or regions on linkage disequilibrium between examined patients and controls from general population, wherein the alteration significantly overrepresented in patients affected by specific malignancy is then regarded as genetic marker being indicative of significantly increased/decreased predisposition to at least one of the following cancers: cancers of the breast, colon, kidney, larynx, lung, pancreas, prostate, thyroid and ovarian.

In the method, the examined structural alteration may be identified by comparison of the structure of the altered CYP1B1 variant with the wild type; and the numbers of examined patients are large enough and results are considered statistically significant when p<0.05.

Also disclosed is a method for analyzing association between CYP1B1 genotypes and predisposition to cancer wherein all homo- and heterozygote variants (combined genotypes) are distinguished.

Applicant herein disclose that 355T/T is associated with predisposition to cancers of the: breast, prostate, larynx, lung and probably thyroid and ovarian. Applicants also disclose the association between 355T/T change in CYP1B1 gene and early onset breast and laryngeal cancers [11].

In September 2005, Cicek et al. published data on 918 sibling based case-control population showing that CYP1B1 355T/T variant is positively associated (OR=3.73 p=0.009) with more aggressive prostate cancer [4]

Further investigation performed by applicants suggest that specificity of above associations can be higher if cases are subclassified by haplotypes determined using two additional polymorphisms resulting in amino acid replacement—R48G and L432V.

There are very few reports on association of haplotypes created using CYP1B1 polymorphic SNPs and predisposition to cancers. Positive association (cancer risk increased or decreased) have been reported only by Chang et al. who indicated that haplotype G-G-C-⁴⁸G¹¹⁹ can be associated with increased risk of prostate cancer and haplotypes T-A-T-G 48-T119 and G⁴⁸-T¹¹⁹-C432 -A453 are associated with decreased risk of prostate cancer [2].

Cicek et al. suggested that CYP1B1 355T-4326C haplotype is positively associated with prostate cancer among men with high aggressive disease (p=0.01) [4].

Genotyping based on CYP1B1 SNPs was not associated with increased risk of breast and endometrial cancers among post-menopausal women in Sweden [12,21]. Similarly, Wen et al. did not observe differences between breast cancers (n=1135) and controls (n=1235) in Shanghai population in frequencies of 8 haplotypes constituted using CYP1B1 R48G-A119S-L432V. It is highly probable that they and others achieved biased results due to application of the expectation-maximization algorithm (described by Hawley et al. in J. Hered. 1995;86:409-11) only what does not differentiate homo- and heterozygote haplotype. With the latest stratification they should asses 27 and not just 8 haplotypes.

TABLE A
List of CYP1B1 variants associated with increased risk of cancers

| Combined genotype | | | |
|---|---|---|---|
| 432 | 48 | 119 | Cancer site |
| C/C | G/G | T/T | Breast, colon, larynx, lung, pancreas, prostate |
| C/C | C/C | G/T | Breast, colon |
| C/C | G/C | T/T | Larynx |
| C/C | G/G | G/G | Colon |
| G/C | G/G | G/T | Prostate |
| G/C | C/C | G/G | Breast |
| G/C | C/C | G/T | Breast, colon |
| G/G | C/C | G/G | Kidney |
| C/C | | T/T | Breast, larynx, pancreas, prostate |
| C/C | | G/T | Lung |
| G/G | | G/G | Kidney |
| G/G | | T/T | Colon |
| C/C | G/G | | Breast, prostate |
| C/C | G/C | | Larynx |
| G/C | C/C | | Breast |
| G/G | C/C | | Kidney |
| | C/C | G/G | Breast, kidney, lung |
| | C/C | G/T | Breast, colon |
| | C/C | T/T | Colon |
| | G/C | T/T | Larynx |
| | G/G | G/G | Colon |
| | G/G | T/T | Breast, prostate |

TABLE B
List of CYP1B1 variants associated with decreased risk of cancers

| Haplotype | | | |
|---|---|---|---|
| 432 | 48 | 119 | Cancer site |
| C/C | G/G | G/G | Breast |
| C/C | G/C | G/G | Breast, prostate |
| C/C | G/G | G/T | Breast |
| G/C | G/C | G/G | Breast, larynx, lung, prostate, |
| G/G | C/C | G/T | Breast |
| G/G | G/C | G/T | Breast, lung, prostate |
| G/G | | T/T | Breast |
| G/G | | G/T | Breast, larynx, lung, pancreas, prostate |
| G/C | | T/T | Breast |
| G/C | | G/G | Colon |
| G/G | G/C | | Breast, larynx, lung, pancreas |
| G/C | G/C | | Breast, prostate |
| | C/C | G/T | lung |
| | G/C | G/G | Breast, larynx, lung, prostate |
| | G/G | G/G | Prostate |
| | G/G | G/T | Breast, larynx |

For the needs of this invention, germline change within CYP1B1 gene is understood as inherited CYP1B1 homozygous 355T/T or other variant (presented in Table A and B) causing production of CYP1B1 protein with activity altered significantly and/or production of protein with altered sequence and potentially altered function, what is related with significantly increased/decreased risk of cancer.

The A119S 355T/T and other (presented in Table A and B) variant alterations are germline changes causing production of improper variants of CYP1B1 protein [12,19]. 355 T/T homozygotes have protein with serine in 119 codon in both of alleles, 142 C/C homozygotes have protein with arginine in 48 codon in both of alleles and 4326 C/C homozygous have protein with leucine in codon 432 in both of alleles. This invention refers to the increased/decreased risk of cancers of various sites in patients with T/T genotype at 355 or other variants (presented in Table A and B) of CYP1B1 gene.

Invention covers also other CYP1B1 alterations linked to 355 T/T and other (presented in Table A and B) variants. Occurrence of A119S 355T/T and other (presented in Table A and B) variants of CYP1B1 gene is examined as an example of application of mode according to invention.

It can be assumed that because different CYP1B1 changes have been reported as associated with cancer predisposition in several populations, 355 T/T and other (presented in Table A and B) variants will also be associated with significantly increased/decreased multiorgane predisposition to malignancies occurring not only among persons of Polish origin but also in other ethnic groups.

In particular realization of the mode according to invention, the presence of germline change is examined at the level of DNA, RNA or protein. The DNA can be examined for example using a technique chosen between RFLP, ASA, microarrays and sequencing.

According to invention, there is also possible to suggest and, then, experimentally verify, the other forms of CYP1B1 DNA, RNA and protein analogous to CYP1B1 forms produced as a result of A119S, R48G and L432V changes. It is recommended to include genetic alterations which are in linkage disequilibrium with 355T/T and other (presented in Table A and B) variants. In case of naturally occurring other variants of CYP1B1 gene/protein, experimental verification of their involvement in carcinogenesis will be based on analysis of associations as it is described in enclosed examples.

The next subject of invention is also the composition for detection of CYP1B1 germline changes allowing identification of person with significantly increased/decreased genetic predisposition to cancers of at least one of the following sites: breast, colon, kidney, larynx, lung, pancreas, prostate and also, most probably, thyroid and ovarian provided 355 T/T or other (presented in Table A and B) variants or changes with analogous features are studied.

The DNA, RNA and proteins can be used as material for diagnostic analyses. For diagnostic purposes it is appropriate to use also altered forms of CYP1B1 DNA/RNA as well as proteins coded by corresponding alleles with alterations. The next subject of invention is thus the use of polypeptide coded by the CYP1B1 allele containg germline variants A119S or other (presented in Table A and B) alterations in linkage disequilibrium with above variants or changes with analogous features for detection of significantly increased/decreased genetic predisposition to cancers of at least one of the following sites: breast, colon, kidney, larynx, lung, pancreas, prostate and, with high probability, thyroid and ovarian.

According to invention the presence of polypeptide coded by CYP1B1 allele with germline alteration is detected by antibodies or other substances specific for this polypeptide or its fragment.

Position of change is not determined by numbers of consecutive nucleotides or aminoacids. According to invention position of a given nucleotide or aminoacid can be marked with different numbers but has to possess the same properties.

It can be particularly valuable when antibodies or other substances specific for such polypeptide or its fragment recognize epitope containing 355T/T or characteristic CYP1B1 protein structure based on variants presented in Table A and B.

Antibodies can be obtained, for example, by a method described by Kohler and Milstein, Nature 256 (1975)7 495, and in Meth. Enzymol. 73 (1981)9 3. Antibodies can be monoclonal, polyclonal or fragments of the antibodies (such as Fab, Fv or scFv) can be used. They can be obtained by methods described by Harlow and Lane “Antibodies, A Laboratory Manual”, CSH Press, Cold Spring Harbor, 1988.

The next subject of invention is composition for detection of significantly increased/decreased genetic predisposition to tumors using techniques based on PCR and characterized by the use of at least two different primers allowing amplification of region containing germline change CYP1B1 A119S or other variant (presented in Table A and B) or alterations in linkage disequilibrium with these variants or with analogous properties what allows detection of predisposition to cancers of the following sites: breast, colon, kidney, larynx, lung, pancreas, prostate and, most probably, thyroid and ovarian.

The next subject of invention is application of polynucleotide containing on position corresponding to position 355 or 142 or 4326 of exon 2/3 of human gene CYP1B1 the change 355 G>T or 142 G>C or 4326A>G or distinct change with analogous properties or other polynucleotide coding CYP1B1 protein variant containing A119S or R48G or L432V substitution (or other with analogous properties) or polypeptide coded by above polynucleotides or antibodies specific for above polypeptides, for preparation of diagnostic composition allowing detection of significantly increased/decreased predisposition to cancers in person with CYP1B1 355 alterations coding A119S substitution or 142 alterations coding R48G substitution or 432 alterations coding L432V substitution change with analogous properties. Above predisposition is associated with increased/decreased risk of cancers of the following sites: breast, colon, kidney, larynx, lung, pancreas, prostate and, most probably, thyroid and ovarian.

One of possible example of above polynucleotide is a polynucleotide obtained using diagnostic composition according to invention; examples of above polypeptide are CYP1B1 polypeptide variants coded by these polynucleotides and corresponding to changes presented in Table A and B.

Definition “corresponding” applied in this description means, that position is not determined by numbers of consecutive nucleotides or aminoacid. According to invention, position of particular nucleotide or aminoacid which can be deleted or substituted, may be distinct than in wild type gene or polypeptide. Thus, “corresponding” position according to invention means that nucleotides or aminoacid may be described using different numbers, but still have the same properties. Such nucleotides or aminoacids which can be substituted or deleted are also covered by definition of “corresponding” position. Such nucleotides or aminoacids can, for example, be part of structures playing crucial role in regulation of gene expression, RNA stability or edition, as well as coding functional domains, splice sites or characteristic motifs of CYP1B1 protein variants or its derivatives.

The next example of invention is application of digestion enzymes which cut allele containing nucleotide (T or G) in 355 or (C or G) in 142 or (C or G) in 432 nucleotide site of CYP1B1 gene e.g. (Earn 11051, PdiI and OliI AvaI-Fermentas) for production of diagnostic composition allowing detection of significantly increased/decreased genetic predisposition to cancers of at least one of the following sites: breast, colon, kidney, larynx, lung, pancreas, prostate and, with high probability, thyroid and ovarian.

The next subject of invention is the mode of identification of genetic marker associated with significantly increased/decreased risk of malignancy development based upon evaluation of biological samples (DNA, RNA or protein) from patients affected by malignancy for the presence of germline 355T/T or other (presented in Table A and B) variants of CYP1B1 or other alterations in linkage disequilibrium with these variant changes. The incidence is then compared to frequency of occurrence of a given variant in general population. The variant with significant overrepresentation/underrepresentation among patients with a given malignancy is considered as a genetic marker associated with significantly increased/decreased risk of malignancy development. It is valuable if the numbers of examined patients are large enough and results are considered statistically significant when p<0.05.

The next subject of invention is the mode of identification of genetic marker associated with significantly increased/decreased risk of malignancy based on analyses of molecular data in all possible combination of genotypes. Such data are much more accurate giving higher or lower risk and diagnostically more valuable than those achived by widely applied haplotyping based on statistical programmes.

The invention will now be described by reference to the following biological examples which are merely illustrative and are not to be construed as a limitation of the scope of the present invention.

### EXAMPLE 1

Studies of Correlation Between CYP1B1 Germline Changes and Predisposition to Cancers of Various Sites

Patients

To establish the range of cancer types associated with 355 variant T/T change we genotype 6605 cases of malignancies of different organs and 3872 of controls in Poland. All cases were unselected and histopatologically confirmed. No selection criteria such as age, sex or cancer family history were used. For genotyping analysis of 142 C<G, 355 G<T and 432 C<G changes we used control group consisted of 1412 newborns born in 2003-2004 in 8 hospitals throughout Poland (Szczecin, Bialystok, Gorzów, Katowice, Wroclaw, Poznań, Lódź i Rzeszów), 854 adults from the region of Szczecin (unselected for the occurrence of malignancies among relatives, male/female ratio 1:1) and group of 450 women matched to age of breast cancer patients (total number of control—2716).

DNA Isolation

5 ml peripheral blood was obtained from patients and mixed with 100 μl 1M EDTA, then was centrifuged in 50 ml polypropylene tubes by 10 minutes at 3000 g in 4° C. Serum in upper faze was removed, and pellet containing cells was mixed with 45 ml buffer 2× (0.1M NH₄Cl, 0.25M KHCO₃, 1 mM EDTA) and was left for 15 minutes in 4° C. Then mixture was centrifuged at 3000 g for 10 minutes in 4° C. Supernatant was removed after centrifugation. The remaining pellet with leukocytes was suspended in 2× buffer and centrifuged 10 minutes at 3000 g in 4° C. This purification of leukocytes in 2× buffer and centrifugation was repeated three times until pure leukocyte pellet was obtained. Then to leukocytes were mixed with 3 ml digestion buffer (50 mM NaCl, 25 mM MgCl₂, 1 mM EDTA; pH 8.0) with 200 μl 10% SDS and 500 μg Proteinase K. Digestion was carried out 24 h in 37° C.

DNA was purified using phenol/chloroform. In brief digestion products was mixed with 3 ml phenol buffered 0.5M Tris HCl (pH 8.4), and then 3 ml chloroform and isoamyl alcohol mixture (mixed in proportion 1:25 vol/vol). Mixture was agitated for about 1 minute and centrifuged 10 minutes at 8000 g in 20° C. After centrifugation upper faze was replaced to new tube, and mixed with equal volume of chloroform and thereafter centrifuged 10 minutes at 8000 g. Above described purification with chloroform was repeated 3-times until protein ring in interfaze had disappeared.

The purified water phase containing DNA was mixed with 5M NaCl in proportion 10:1 (vol/vol) and 96% ethanol in the proportion of water faze with NaCl to ethanol 1:10 (vol/vol). Mixture was left overnight in 20° C. The resultant DNA pellet was placed in a new tube and purified with 70% ethanol, centrifuged at 3000 g for 5 minutes, and ethanol was poured out. Then purified DNA pellet was dried in open tube for 30 minutes at 37° C. DNA resuspended in 400 μl TE buffer (25 mM Tris HCl, 1 mM EDTA; pH 8.4) was stored in 4° C. until use.

Restriction Fragment Length Polymorphism Polymerase Chain Reaction (RFLP-PCR)

Variants 355T>G

The 355T/T variant alteration was identified by RFLP-PCR using Eam 1105I and PdiI restriction enzyme (Fermentas). PCR was performed with primers e.g. CYP119F (CTCGTTCGCTCGCCTGGCGC) (SEQ ID NO.:1) and e.g. CYP119R (GAAGTTGCGCATCATGCTGT) (SEQ ID NO.:2). PCR reactions was carried out in PTC-200 Peltier DNA ThermalCycler (MJ Research) in volume of 25 l included: 1 μl (50 ng) genomic DNA, 4 pmol each primer set, 2.5 μl PCR Buffer 2(Expand Long Template PCR System Roche—22.5 mM MgCl₂), 200 μM each dATP, dCTP, dGTP i dTTP and 1 U Taq DNA polymerase. In each reaction negative control (control without DNA) was used.

PCR Conditions:

a) Initial denaturation −95° C. 15 minutes
b) 15 cycles, each of: denaturation −95° C. 30 s

primer annealing −62-54.5° C. 30s
(decrease temperature 0.5° C. in each cycle)

primer elongation −72° C. 2minutes
c) 21 cycles, each of: denatution −95° C. 30 s

primer annealing −57° C. 30 s

primer elongation −72° C. 30 s
d) elongation −72° C. 8 minutes

Digestion was performed overnight at 37° C. in volume of 24 μl containing: 12 μl PCR product, 10× Buffer Tango (Fermentas) and 2 U Eam 1105I enzyme (Fermentas). Then, 15 μl of digestion product was mixed with 10 μl loading buffer and was electrophoresed in agarose gel (3% agarose gel (SeaKem FMC), 1× bufor TBE, 25 μg/ml ethidium bromide) at 6V/cm for 30 minutes. Separated PCR products were visualized in UV light. PCR product (250 bp) was digested on two fragments: 136 bp and 114 bp in cases which containing nucleotide T in 355 nucleotide site of CYP1B1 gene. All cases with alterations are verified by using PdiI enzyme restriction (Fermentas). Restriction mixture in volume 18 μl containing: 4 μl PCR product, 10× Buffer Tango (Fermentas) and 2 U PdiI enzyme (Fermentas). Then, 15 μl digestion product was electophoresed in the same conditions. PCR product (250 bp) was digested on two fragments: 138 bp and 112 bp in cases which containing nucleotide G in 355 nucleotide site of CYP1B1 gene. In addition, randomly selected cases with G/G, T/T and G/T variants were sequenced in order to confirm the presence of the A119S change. Sequencing was prepared by using conventional methods.

Purification of PCR Product

Sequencing product were placed on Microcon—100 (Amicon) column which fit on 0.5 ml Eppendorf tube. 400 μl distillated water was added, then centrifuged for 15 minutes at 1850 g in 25° C. The columns were 4 times washed with 400 μl of distillated water. After the last washing step, the columns were turned up side down and placed on a new Eppendorf tube. By centrifugation for 3 minutes at 9000 g we obtain 5 μl purified PCR product which were 4 times diluted with distillated water.

Variants 142 G>C

The variants of R48G alteration was identified by RFLP-PCR using Eco88I (AvaI) restriction enzyme (Fermentas). PCR was performed with primers e.g. F1CYP (TCCATCCAGCAGACCACGCT) (SEQ ID NO.:3) and e.g. R1 (GCCGGACACCACACGGAAG) (SEQ ID NO.:4).

PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2(Expand Long Template PCR System Roche—22.5 mM MgCl₂), 200 μM each dATP, dCTP, dGTP i dTTP and 1 U Taq DNA polymerase. In each reaction negative control (control without DNA) was used.

PCR Conditions:

b) Initial denaturation −95° C. 5 minutes
b) 29 cycles, each of: denaturation −94° C. 30 s

primer annealing −56 ° C. 30s
primer elongation −72° C. 30s
c) elongation −72° C. 5 minutes

Digestion was performed overnight at 37° C. in volume of 24 μl containing: 12 μl PCR product, 10× Buffer Tango (Fermentas) and 2 U Eco88I (AvaI) enzyme (Fermentas). Then, 15 μl of digestion product was mixed with 10 μl loading buffer and was electrophoresed in agarose gel (4% agarose gel (SeaKem FMC), 1× bufor TBE, 25 μg/ml ethidium bromide) at 6V/cm for 30 minutes. Separated PCR products were visualized in UV light. PCR product (336 bp) was digested on three fragments: 14 bp, 91 bp and 230 bp in cases which containing nucleotide G in 142 nucleotide site of CYP1B1 gene. In addition, randomly selected cases with G/G, C/C and C/G variants were sequenced in order to confirm the presence of the R48G change. Sequencing was prepared by using conventional methods.

Purification of PCR Product

Sequencing product were placed on Microcon—100 (Amicon) column which fit on 0.5 ml Eppendorf tube. 400 μl distillated water was added, then centrifuged for 15 minutes at 1850 g in 25° C. The columns were 4 times washed with 400 μl of distillated water. After the last washing step, the columns were turned up side down and placed on a new Eppendorf tube. By centrifugation for 3 minutes at 9000 g we obtain 5 μl purified PCR product which were 4 times diluted with distillated water.

Variants 432 C>G

The variants of L432V alteration was identified by RFLP-PCR using OliI restriction enzyme (Fermentas). PCR was performed with primers e.g. CYP1294F (ATGCGCTTCTCCAGCTTTGT) (SEQ ID NO.:5) and e.g. CYP1294R (TATGGAGCACACCTCACCTG) (SEQ ID NO.:6).

PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2(Expand Long Template PCR System Roche—22.5 mM MgCl₂), 200 μM each dATP, dCTP, dGTP i dTTP and 1 U Taq DNA polymerase. In each reaction negative control (control without DNA) was used.

PCR Conditions:

c) Initial denaturation −95° C. 15 minutes
b) 10 cycles, each of: denaturation −95° C. 30 s

primer annealing −62-57° C. 30s
(decrease temperature 0.5° C. in each cycle)

primer elongation −72° C. 2minutes
d) 30 cycles, each of: denatution −95° C. 30 s

primer annealing −57° C. 30 s

primer elongation −72° C. 30 s
d) elongation −72° C. 8 minutes

Digestion was performed overnight at 37° C. in volume of 24 μl containing: 12 μl PCR product, 10× Buffer R (Fermentas) and 2U OliI enzyme (Fermentas). Then, 15 μl of digestion product was mixed with 10 μl loading buffer and was electrophoresed in agarose gel (3% agarose gel (SeaKem FMC), 1× bufor TBE, 25 μg/ml ethidium bromide) at 6V/cm for 30 minutes. Separated PCR products were visualized in UV light. PCR product (623 bp) was digested on two fragments: 132 bp and 491 bp in cases which containing nucleotide C in 4329 nucleotide site of CYP1B1 gene. In addition, randomly selected cases with G/G, C/C and C/G variants were sequenced in order to confirm the presence of the L432V change. Sequencing was prepared by using conventional methods.

Purification of PCR Product

Sequencing product were placed on Microcon—100 (Amicon) column which fit on 0.5 ml Eppendorf tube. 400 μl distillated water was added, then centrifuged for 15 minutes at 1850 g in 25° C. The columns were 4 times washed with 400 μl of distillated water. After the last washing step, the columns were turned up side down and placed on a new Eppendorf tube. By centrifugation for 3 minutes at 9000 g we obtain 5 μl purified PCR product which were 4 times diluted with distillated water.

Results

I. Results on 3 Variants 355-T/G, G/G and T/T are Summarized in Tables 1-3.

TABLE 1
The frequency of the 355TT variant in cases and controls

| | | No of subject | | | |
|---|---|---|---|---|---|
| | No of | with T/T | | | |
| Cancer | subject | variant | Odds | | CI-interval |
| site | tested | (frequency %) | ratio | p-value | confidence |
| Bladder | 178 | 15 (8.4%) | 0.9653 | 0.8981 | 0.5622-1.658 |
| Breast | 2084 | 217 (10.4%) | 1.22 | 0.0341 | 1.019-1.459 |
| Colon | 482 | 54 (11.2%) | 1.323 | 0.084 | 0.9762-1.794 |
| Kidney* | 220 | 22 (10%) | 1.166 | 0.5901 | 0.7398-1.836 |
| Larynx | 410 | 50 (12.20%) | 1.457 | 0.0242 | 1.062-1.999 |
| Lung | 474 | 54 (11.39%) | 1.349 | 0.0649 | 0.9945-1.829 |
| Melanoma | 262 | 29 (11.07%) | 1.306 | 0.2333 | 0.8735-1.951 |
| Pancreatic | 240 | 31 (12.91%) | 1.556 | 0.0355 | 1.050-2.306 |
| Ovary | 500 | 39 (7.8%) | 0.8874 | 0.5529 | 0.6282-1.254 |
| Prostate | 1312 | 141 (10.7%) | 1.263 | 0.0311 | 1.026-1.554 |
| Stomach | 232 | 21 (9.05%) | 1.044 | 0.9499 | 0.6575-1.658 |
| Thyroid** | 211 | 23 (10.90%) | 1.283 | 0.3314 | 0.8205-2.007 |
| Control | 3872 | 337 (8.7%) | | | |
| total | | | | | |
| Newborns | 2091 | 186 (8.8%) | | | |
| Adults | 1295 | 109 (8.4%) | | | |
| Matched | 486 | 42 (8.6%) | | | |
| women | | | | | |

*renal clear cell carcinoma,
**papillary type

TABLE 2
The frequency of the 355 GG variant in cases and controls

| | | No of subject | | | |
|---|---|---|---|---|---|
| | No of | with G/G | | | |
| Cancer | subject | variant | Odds | | CI-interval |
| site | tested | (frequency %) | ratio | p-value | confidence |
| Bladder | 178 | 81 | 0.9222 | 0.6529 | 0.6820-1.247 |
| Breast | 2084 | 965 | 0.9524 | 0.3848 | 0.8559-1.060 |
| Colon | 482 | 216 | 0.8968 | 0.2826 | 0.7413-1.085 |
| Kidney* | 220 | 111 | 1.125 | 0.4365 | 0.8570-1.476 |
| Larynx | 410 | 182 | 0.8815 | 0.2479 | 0.7183-1.082 |
| Lung | 474 | 218 | 0.9404 | 0.5615 | 0.7766-1.139 |
| Melanoma | 262 | 117 | 0.8911 | 0.4039 | 0.6928-1.146 |
| Pancreatic | 240 | 102 | 0.8163 | 0.1484 | 0.6271-1.062 |
| Ovary | 500 | 260 | 1.196 | 0.0659 | 0.9928-1.442 |
| Prostate | 1312 | 595 | 0.9164 | 0.1838 | 0.8082-1.039 |
| Stomach | 232 | 98 | 0.807 | 0.1344 | 0.6177-1.056 |
| Thyroid** | 211 | 84 | 0.7304 | 0.0345 | 0.5505-0.969 |
| Control | 3872 | 1840 | | | |
| total | | | | | |
| Newborns | 2091 | 989 | | | |
| Adults | 1295 | 611 | | | |
| Matched | 486 | 240 | | | |
| women | | | | | |

*renal clear cell carcinoma,
**papillary type,

TABLE 3
The frequency of the 355 GT variant in cases and controls

| | | No of subject | | | |
|---|---|---|---|---|---|
| | No of | with G/T | | | |
| Cancer | subject | variant | Odds | | CI-interval |
| site | tested | (frequency %) | ratio | p-value | confidence |
| Bladder | 178 | 82 | 0.9433 | 0.7620 | 0.6978-1.275 |
| Breast | 2084 | 902 | 0.8427 | 0.0019 | 0.7571-0.938 |
| Colon | 482 | 212 | 0.8671 | 0.1560 | 0.7166-1.049 |
| Kidney* | 220 | 94 | 0.8239 | 0.1882 | 0.6261-1.084 |
| Larynx | 410 | 178 | 0.8473 | 0.1256 | 0.6901-1.040 |
| Lung | 474 | 202 | 0.8201 | 0.0488 | 0.6764-0.994 |
| Melanoma | 262 | 116 | 0.8774 | 0.3398 | 0.6821-1.129 |
| Pancreatic | 240 | 107 | 0.888 | 0.4135 | 0.6835-1.155 |
| Ovary | 500 | 201 | 0.8634 | 0.01414 | 0.7142-1.044 |
| Prostate | 1312 | 576 | 0.8643 | 0.0252 | 0.7620-0.980 |
| Stomach | 232 | 113 | 1.049 | 0.7766 | 0.8044-1.367 |
| Thyroid** | 211 | 104 | 1.073 | 0.6671 | 0.8135-1.416 |
| Control | 3872 | 1695 | | | |
| total | | | | | |
| Newborns | 2091 | 916 | | | |
| Adults | 1295 | 575 | | | |
| Matched | 486 | 204 | | | |
| women | | | | | |

*renal clear cell carcinoma
**papillary type

Significantly increased frequency of 355T/T variant was observed for breast, larynx, pancreas and prostate cancer cases. Some additional correlations have been found when cases were classified into subgroups depending on age at diagnosis—355 T/T variant was also more frequent in group of papillary cancer of the thyroid for subgroup diagnosed between 40-49 years. The strongest association between 355T/T variant and cancer risk was identified for tumors diagnosed at age 50-59 years for cancers of the larynx, diagnosed at age >59 for prostate cancers and <40 years for breast cancer. Value of data on laryngeal cancers has been emphasized by results of our separate analyses of clinical features of T/T positive patients diagnosed at age 50-59 years—their cancers have been shown to be more aggressive morphologically and clinically (Jaworowska et al.2005, submitted).

Additionally, we found association between 355T/T variant and ovarian cancer risk for sub-groups of tumours diagnosed at age above 50 and with G3 morphological grade. Presented results indicated that A119S variant T/T is pathogenic alteration which confers increased risk of breast, larynx, prostate, pancreas and, probably, thyroid and ovarian cancer. It cannot be ruled out that A119S alteration predisposes to additional malignancies. Larger registries of patients are needed in order to evaluate these associations.

On contrary to 355 T/T, 355 G/G and G/T have been showing effects preventing against cancers—355 G/G carriers have decreased risk of thyroid cancer and 355G/T decreased risk of cancers of the breast, lung and prostate.

II. Results on Combined Genotypes-Variants Including Alternative SNPs at Three Codons—48,119 and 432. The Above Results are Presented in Tables 4-11.

All of combined genotypes associated with increased or decreased risk of cancers are also listed in Table A and B.

TABLE 4
Breast cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 121 | 6.55% | 163 | 6.00% | 0.472429 | 0.49187172 | | 1.097378 |
| | | G/T | 34 | 1.84% | 23 | 0.85% | 8.005688 | 0.00466306 | p < 0.05 | 2.194574 |
| | | T/T | 2 | 0.11% | 6 | 0.22% | 0.284008 | 0.59408628 | | 0.489346 |
| | G/C | G/G | 7 | 0.38% | 47 | 1.73% | 16.049 | 0.00006172 | p < 0.05 | 0.215921 |
| | | G/T | 252 | 13.64% | 346 | 12.74% | 0.700438 | 0.40263657 | | 1.081533 |
| | | T/T | 15 | 0.81% | 10 | 0.37% | 3.198289 | 0.07371536 | | 2.214403 |
| | G/G | G/G | 1 | 0.05% | 13 | 0.48% | 5.167369 | 0.02301505 | p < 0.05 | 0.112573 |
| | | G/T | 6 | 0.32% | 33 | 1.22% | 9.265851 | 0.00233466 | p < 0.05 | 0.264831 |
| | | T/T | 182 | 9.85% | 180 | 6.63% | 15.18698 | 0.00009737 | p < 0.05 | 1.539122 |
| G/C | C/C | G/G | 436 | 23.59% | 521 | 19.18% | 12.64402 | 0.00037677 | p < 0.05 | 1.300914 |
| | | G/T | 61 | 3.30% | 60 | 2.21% | 4.664471 | 0.03079291 | p < 0.05 | 1.511061 |
| | | T/T | | 0.00% | 5 | 0.18% | 1.931271 | 0.16461957 | | 0 |
| | G/C | G/G | 8 | 0.43% | 83 | 3.06% | 37.39105 | 0.00000000 | p < 0.05 | 0.137926 |
| | | G/T | 422 | 22.84% | 643 | 23.67% | 0.387116 | 0.53381934 | | 0.954072 |
| | | T/T | | 0.00% | 7 | 0.26% | 3.235655 | 0.07205149 | | 0 |
| | G/G | G/G | 8 | 0.43% | 8 | 0.29% | 0.271593 | 0.60226541 | | 1.471739 |
| | | G/T | 3 | 0.16% | 8 | 0.29% | 0.344192 | 0.55741962 | | 0.550407 |
| | | T/T | 4 | 0.22% | 13 | 0.48% | 1.391996 | 0.23806840 | | 0.451026 |
| G/G | C/C | G/G | 266 | 14.39% | 427 | 15.72% | 1.403985 | 0.23605759 | | 0.901349 |
| | | G/T | 3 | 0.16% | 21 | 0.77% | 6.720636 | 0.00953036 | p < 0.05 | 0.208672 |
| | | T/T | 1 | 0.05% | 3 | 0.11% | 0.014862 | 0.90297077 | | 0.489623 |
| | G/C | G/G | 1 | 0.05% | 20 | 0.74% | 9.736879 | 0.00180607 | p < 0.05 | 0.072983 |
| | | G/T | 12 | 0.65% | 62 | 2.28% | 17.38498 | 0.00003052 | p < 0.05 | 0.279781 |
| | | T/T | | 0.00% | 2 | 0.07% | 0.199263 | 0.65531610 | | 0 |
| | G/G | G/G | 2 | 0.11% | 4 | 0.15% | 0.003447 | 0.95317901 | | 0.734561 |
| | | G/T | 1 | 0.05% | 1 | 0.04% | 0.199263 | 0.65531610 | | 1.469951 |
| | | T/T | | 0.00% | 7 | 0.26% | 3.235655 | 0.07205149 | | 0 |
| Total | | | 1848 | 100.00% | 2716 | 100.00% | | | | |

TABLE 5
Colon cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 21 | 5.90% | 163 | 6.00% | 0.001769 | 0.96644756 | | 0.981833 |
| | | G/T | 8 | 2.25% | 23 | 0.85% | 4.85641 | 0.02754360 | p < 0.05 | 2.691654 |
| | | T/T | 1 | 0.28% | 6 | 0.22% | 0.135341 | 0.71295755 | | 1.2723 |
| | G/C | G/G | 10 | 2.81% | 47 | 1.73% | 1.461767 | 0.22664928 | | 1.64125 |
| | | G/T | 45 | 12.64% | 346 | 12.74% | 0.00102 | 0.97452711 | | 0.991116 |
| | | T/T | 2 | 0.56% | 10 | 0.37% | 0.009768 | 0.92126966 | | 1.528814 |
| | G/G | G/G | 8 | 2.25% | 13 | 0.48% | 12.0122 | 0.00052854 | p < 0.05 | 4.779841 |
| | | G/T | 2 | 0.56% | 33 | 1.22% | 0.682943 | 0.40857518 | | 0.459339 |
| | | T/T | 27 | 7.58% | 180 | 6.63% | 0.318956 | 0.57223567 | | 1.156231 |
| G/C | C/C | G/G | 55 | 15.45% | 521 | 19.18% | 2.639501 | 0.10423629 | | 0.769827 |
| | | G/T | 17 | 4.78% | 60 | 2.21% | 7.463982 | 0.00629456 | p < 0.05 | 2.219862 |
| | | T/T | 1 | 0.28% | 5 | 0.18% | 0.062176 | 0.80308966 | | 1.527324 |
| | G/C | G/G | 8 | 2.25% | 83 | 3.06% | 0.462499 | 0.49645855 | | 0.729262 |
| | | G/T | 81 | 22.75% | 643 | 23.67% | 0.101683 | 0.74981973 | | 0.9496 |
| | | T/T | | 0.00% | 7 | 0.26% | 0.135341 | 0.71295755 | | 0 |
| | G/G | G/G | 1 | 0.28% | 8 | 0.29% | 0.227188 | 0.63361647 | | 0.953521 |
| | | G/T | 1 | 0.28% | 8 | 0.29% | 0.227188 | 0.63361647 | | 0.953521 |
| | | T/T | 1 | 0.28% | 13 | 0.48% | 0.010492 | 0.91841563 | | 0.585699 |
| G/G | C/C | G/G | 52 | 14.61% | 427 | 15.72% | 0.218586 | 0.64011907 | | 0.916954 |
| | | G/T | 1 | 0.28% | 21 | 0.77% | 0.492163 | 0.48296401 | | 0.361502 |
| | | T/T | 8 | 2.25% | 3 | 0.11% | 34.50984 | 0.00000000 | p < 0.05 | 20.78927 |
| | G/C | G/G | 2 | 0.56% | 20 | 0.74% | 0.001094 | 0.97361458 | | 0.761582 |
| | | G/T | 2 | 0.56% | 62 | 2.28% | 3.765023 | 0.05233516 | | 0.241844 |
| | | T/T | | 0.00% | 2 | 0.07% | 0.35134 | 0.55335561 | | 0 |
| | G/G | G/G | 2 | 0.56% | 4 | 0.15% | 1.055395 | 0.30426812 | | 3.830508 |
| | | G/T | | 0.00% | 1 | 0.04% | 1.440541 | 0.23005193 | | 0 |
| | | T/T | | 0.00% | 7 | 0.26% | 0.135341 | 0.71295755 | | 0 |
| Total | | | 356 | 100.00% | 2716 | 100.00% | | | | |

TABLE 6
Kidney cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 11 | 5.47% | 163 | 6.00% | 0.022845 | 0.87986017 | | 0.906781 |
| | | G/T | 3 | 1.49% | 23 | 0.85% | 0.303571 | 0.58165247 | | 1.774045 |
| | | T/T | | 0.00% | 6 | 0.22% | 0.019505 | 0.88892916 | | 0 |
| | G/C | G/G | | 0.00% | 47 | 1.73% | 2.527908 | 0.11184842 | | 0 |
| | | G/T | 27 | 13.43% | 346 | 12.74% | 0.030505 | 0.86135041 | | 1.062886 |
| | | T/T | | 0.00% | 10 | 0.37% | 0.055906 | 0.81308815 | | 0 |
| | G/G | G/G | 1 | 0.50% | 13 | 0.48% | 0.241565 | 0.62307784 | | 1.039615 |
| | | G/T | 1 | 0.50% | 33 | 1.22% | 0.329479 | 0.56596615 | | 0.406515 |
| | | T/T | 20 | 9.95% | 180 | 6.63% | 2.736284 | 0.09809266 | | 1.556783 |
| G/C | C/C | G/G | 41 | 20.40% | 521 | 19.18% | 0.108182 | 0.74222324 | | 1.079595 |
| | | G/T | 4 | 1.99% | 60 | 2.21% | 0.002016 | 0.96418311 | | 0.898816 |
| | | T/T | | 0.00% | 5 | 0.18% | 0.075475 | 0.78352571 | | 0 |
| | G/C | G/G | | 0.00% | 83 | 3.06% | 5.265247 | 0.02175534 | p < 0.05 | 0 |
| | | G/T | 44 | 21.89% | 643 | 23.67% | 0.239145 | 0.62482400 | | 0.903527 |
| | | T/T | | 0.00% | 7 | 0.26% | 0.000696 | 0.97895705 | | 0 |
| | G/G | G/G | | 0.00% | 8 | 0.29% | 0.005132 | 0.94289181 | | 0 |
| | | G/T | | 0.00% | 8 | 0.29% | 0.005132 | 0.94289181 | | 0 |
| | | T/T | 1 | 0.50% | 13 | 0.48% | 0.241565 | 0.62307784 | | 1.039615 |
| G/G | C/C | G/G | 48 | 23.88% | 427 | 15.72% | 8.550138 | 0.00345496 | p < 0.05 | 1.681774 |
| | | G/T | | 0.00% | 21 | 0.77% | 0.670498 | 0.41287812 | | 0 |
| | | T/T | | 0.00% | 3 | 0.11% | 0.447342 | 0.50359983 | | 0 |
| | G/C | G/G | | 0.00% | 20 | 0.74% | 0.605091 | 0.43664208 | | 0 |
| | | G/T | | 0.00% | 62 | 2.28% | 3.654892 | 0.05590480 | | 0 |
| | | T/T | | 0.00% | 2 | 0.07% | 1.023013 | 0.31180569 | | 0 |
| | G/G | G/G | | 0.00% | 4 | 0.15% | 0.19644 | 0.65761003 | | 0 |
| | | G/T | | 0.00% | 1 | 0.04% | 2.897604 | 0.08871133 | | 0 |
| | | T/T | | 0.00% | 7 | 0.26% | 0.000696 | 0.97895705 | | 0 |
| Total | | | 201 | 100.00% | 2716 | 100.00% | | | | |

TABLE 7
Larynx cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 22 | 6.23% | 163 | 6.00% | 0.002762 | 0.95808661 | | 1.041017 |
| | | G/T | | 0.00% | 23 | 0.85% | 1.980981 | 0.15928717 | | 0 |
| | | T/T | 1 | 0.28% | 6 | 0.22% | 0.13098 | 0.71741817 | | 1.283144 |
| | G/C | G/G | 2 | 0.57% | 47 | 1.73% | 2.00376 | 0.15690959 | | 0.323574 |
| | | G/T | 58 | 16.43% | 346 | 12.74% | 3.407786 | 0.06488947 | | 1.346723 |
| | | T/T | 9 | 2.55% | 10 | 0.37% | 20.74551 | 0.00000525 | p < 0.05 | 7.079651 |
| | G/G | G/G | | 0.00% | 13 | 0.48% | 0.751754 | 0.38592135 | | 0 |
| | | G/T | 1 | 0.28% | 33 | 1.22% | 1.698017 | 0.19254755 | | 0.230975 |
| | | T/T | 35 | 9.92% | 180 | 6.63% | 4.690537 | 0.03032917 | p < 0.05 | 1.550664 |
| G/C | C/C | G/G | 73 | 20.68% | 521 | 19.18% | 0.357883 | 0.54968449 | | 1.098403 |
| | | G/T | 6 | 1.70% | 60 | 2.21% | 0.181198 | 0.67034582 | | 0.765418 |
| | | T/T | | 0.00% | 5 | 0.18% | 0.011101 | 0.91608785 | | 0 |
| | G/C | G/G | 1 | 0.28% | 83 | 3.06% | 8.009995 | 0.00465199 | p < 0.05 | 0.090122 |
| | | G/T | 84 | 23.80% | 643 | 23.67% | 0.000257 | 0.98721733 | | 1.006735 |
| | | T/T | | 0.00% | 7 | 0.26% | 0.13098 | 0.71741817 | | 0 |
| | G/G | G/G | 1 | 0.28% | 8 | 0.29% | 0.236522 | 0.62672928 | | 0.961648 |
| | | G/T | | 0.00% | 8 | 0.29% | 0.217361 | 0.64105804 | | 0 |
| | | T/T | 1 | 0.28% | 13 | 0.48% | 0.008576 | 0.92621723 | | 0.590691 |
| G/G | C/C | G/G | 54 | 15.30% | 427 | 15.72% | 0.016492 | 0.89781466 | | 0.968145 |
| | | G/T | 1 | 0.28% | 21 | 0.77% | 0.477593 | 0.48951442 | | 0.364583 |
| | | T/T | | 0.00% | 3 | 0.11% | 0.078686 | 0.77908542 | | 0 |
| | G/C | G/G | | 0.00% | 20 | 0.74% | 1.602685 | 0.20552324 | | 0 |
| | | G/T | 2 | 0.57% | 62 | 2.28% | 3.7049 | 0.05425292 | | 0.243911 |
| | | T/T | | 0.00% | 2 | 0.07% | 0.358207 | 0.54950389 | | 0 |
| | G/G | G/G | 2 | 0.57% | 4 | 0.15% | 1.076023 | 0.29958908 | | 3.863248 |
| | | G/T | | 0.00% | 1 | 0.04% | 1.45648 | 0.22749114 | | 0 |
| | | T/T | | 0.00% | 7 | 0.26% | 0.13098 | 0.71741817 | | 0 |
| Total | | | 353 | 100.00% | 2716 | 100.00% | | | | |

TABLE 8
Lung cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 18 | 5.44% | 163 | 6.00% | 0.081947 | 0.77467569 | | 0.900723 |
| | | G/T | 2 | 0.60% | 23 | 0.85% | 0.019394 | 0.88924230 | | 0.711775 |
| | | T/T | | 0.00% | 6 | 0.22% | 0.039735 | 0.84200010 | | 0 |
| | G/C | G/G | 3 | 0.91% | 47 | 1.73% | 0.783474 | 0.37608072 | | 0.519395 |
| | | G/T | 39 | 11.78% | 346 | 12.74% | 0.165702 | 0.68395995 | | 0.914859 |
| | | T/T | 2 | 0.60% | 10 | 0.37% | 0.033335 | 0.85512813 | | 1.644985 |
| | G/G | G/G | | 0.00% | 13 | 0.48% | 0.663879 | 0.41519374 | | 0 |
| | | G/T | | 0.00% | 33 | 1.22% | 3.010697 | 0.08271675 | | 0 |
| | | T/T | 33 | 9.97% | 180 | 6.63% | 4.568476 | 0.03256553 | p < 0.05 | 1.560179 |
| G/C | C/C | G/G | 73 | 22.05% | 521 | 19.18% | 1.372811 | 0.24132957 | | 1.192065 |
| | | G/T | 2 | 0.60% | 60 | 2.21% | 3.049727 | 0.08075071 | | 0.269098 |
| | | T/T | | 0.00% | 5 | 0.18% | 0.003853 | 0.95050290 | | 0 |
| | G/C | G/G | 3 | 0.91% | 83 | 3.06% | 4.217849 | 0.04000084 | p < 0.05 | 0.290148 |
| | | G/T | 92 | 27.79% | 643 | 23.67% | 2.515796 | 0.11271075 | | 1.241018 |
| | | T/T | | 0.00% | 7 | 0.26% | 0.100285 | 0.75148763 | | 0 |
| | G/G | G/G | | 0.00% | 8 | 0.29% | 0.176284 | 0.67458634 | | 0 |
| | | G/T | | 0.00% | 8 | 0.29% | 0.176284 | 0.67458634 | | 0 |
| | | T/T | 3 | 0.91% | 13 | 0.48% | 0.376657 | 0.53939776 | | 1.901735 |
| G/G | C/C | G/G | 60 | 18.13% | 427 | 15.72% | 1.098288 | 0.29464235 | | 1.186861 |
| | | G/T | | 0.00% | 21 | 0.77% | 1.571181 | 0.21003576 | | 0 |
| | | T/T | | 0.00% | 3 | 0.11% | 0.104453 | 0.74655030 | | 0 |
| | G/C | G/G | | 0.00% | 20 | 0.74% | 1.454174 | 0.22785952 | | 0 |
| | | G/T | 1 | 0.30% | 62 | 2.28% | 4.779886 | 0.02879404 | p < 0.05 | 0.129717 |
| | | T/T | | 0.00% | 2 | 0.07% | 0.413055 | 0.52042293 | | 0 |
| | G/G | G/G | | 0.00% | 4 | 0.15% | 0.011083 | 0.91615875 | | 0 |
| | | G/T | | 0.00% | 1 | 0.04% | 1.582346 | 0.20842315 | | 0 |
| | | T/T | | 0.00% | 7 | 0.26% | 0.100285 | 0.75148763 | | 0 |
| Total | | | 331 | 100.00% | 2716 | 100.00% | | | | |

TABLE 9
Pancreas cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 13 | 5.73% | 163 | 6.00% | 0.000481 | 0.98250414 | | 0.951465 |
| | | G/T | 3 | 1.32% | 23 | 0.85% | 0.133337 | 0.71499735 | | 1.568129 |
| | | T/T | | 0.00% | 6 | 0.22% | 0.003248 | 0.95455262 | | 0 |
| | G/C | G/G | 3 | 1.32% | 47 | 1.73% | 0.036348 | 0.84879853 | | 0.760543 |
| | | G/T | 28 | 12.33% | 346 | 12.74% | 0.005194 | 0.94254511 | | 0.963778 |
| | | T/T | 3 | 1.32% | 10 | 0.37% | 2.433387 | 0.11877605 | | 3.624107 |
| | G/G | G/G | | 0.00% | 13 | 0.48% | 0.274318 | 0.60045011 | | 0 |
| | | G/T | 1 | 0.44% | 33 | 1.22% | 0.526697 | 0.46799895 | | 0.359748 |
| | | T/T | 25 | 11.01% | 180 | 6.63% | 5.559783 | 0.01837769 | p < 0.05 | 1.743674 |
| G/C | C/C | G/G | 38 | 16.74% | 521 | 19.18% | 0.661296 | 0.41610289 | | 0.847069 |
| | | G/T | 1 | 0.44% | 60 | 2.21% | 2.415819 | 0.12011514 | | 0.19587 |
| | | T/T | | 0.00% | 5 | 0.18% | 0.036795 | 0.84788403 | | 0 |
| | G/C | G/G | 3 | 1.32% | 83 | 3.06% | 1.652072 | 0.19867718 | | 0.42486 |
| | | G/T | 67 | 29.52% | 643 | 23.67% | 3.591862 | 0.05806316 | | 1.350029 |
| | | T/T | | 0.00% | 7 | 0.26% | 0.003207 | 0.95484185 | | 0 |
| | G/G | G/G | | 0.00% | 8 | 0.29% | 0.024128 | 0.87656056 | | 0 |
| | | G/T | | 0.00% | 8 | 0.29% | 0.024128 | 0.87656056 | | 0 |
| | | T/T | 1 | 0.44% | 13 | 0.48% | 0.177982 | 0.67311346 | | 0.920014 |
| G/G | C/C | G/G | 40 | 17.62% | 427 | 15.72% | 0.432837 | 0.51060003 | | 1.146664 |
| | | G/T | | 0.00% | 21 | 0.77% | 0.844846 | 0.35801437 | | 0 |
| | | T/T | | 0.00% | 3 | 0.11% | 0.338197 | 0.56087178 | | 0 |
| | G/C | G/G | | 0.00% | 20 | 0.74% | 0.768826 | 0.38058047 | | 0 |
| | | G/T | 1 | 0.44% | 62 | 2.28% | 2.571501 | 0.10880446 | | 0.189409 |
| | | T/T | | 0.00% | 2 | 0.07% | 0.840192 | 0.35934192 | | 0 |
| | G/G | G/G | | 0.00% | 4 | 0.15% | 0.128933 | 0.71954128 | | 0 |
| | | G/T | | 0.00% | 1 | 0.04% | 2.512938 | 0.11291528 | | 0 |
| | | T/T | | 0.00% | 7 | 0.26% | 0.003207 | 0.95484185 | | 0 |
| Total | | | 227 | 100.00% | 2716 | 100.00% | | | | |

TABLE 10
Prostate cancer

| CYP1B1 | | | Cases | | Control | | | | | |
|---|---|---|---|---|---|---|---|---|---|---|
| variants | | | | Freq. | | Freq. | Statistical analysis | | | |
| 432 | 48 | 119 | No. | (%) | No. | (%) | CHI2 | p-value | | OR |
| C/C | C/C | G/G | 35 | 5.83% | 163 | 6.00% | 0.003859 | 0.95046819 | | 0.970248 |
| | | G/T | 3 | 0.50% | 23 | 0.85% | 0.379473 | 0.53788565 | | 0.588377 |
| | | T/T | | 0.00% | 6 | 0.22% | 0.386414 | 0.53419047 | | 0 |
| | G/C | G/G | 3 | 0.50% | 47 | 1.73% | 4.215991 | 0.04004467 | p < 0.05 | 0.285363 |
| | | G/T | 71 | 11.83% | 346 | 12.74% | 0.289128 | 0.59078012 | | 0.919337 |
| | | T/T | 4 | 0.67% | 10 | 0.37% | 0.452434 | 0.50118125 | | 1.816107 |
| | G/G | G/G | 1 | 0.17% | 13 | 0.48% | 0.516658 | 0.47227080 | | 0.347117 |
| | | G/T | 2 | 0.33% | 33 | 1.22% | 2.862532 | 0.09066471 | | 0.271916 |
| | | T/T | 82 | 13.67% | 180 | 6.63% | 32.50387 | 0.00000001 | p < 0.05 | 2.230287 |
| G/C | C/C | G/G | 119 | 19.83% | 521 | 19.18% | 0.095088 | 0.75780595 | | 1.042314 |
| | | G/T | 9 | 1.50% | 60 | 2.21% | 0.889809 | 0.34552895 | | 0.674112 |
| | | T/T | 2 | 0.33% | 5 | 0.18% | 0.052628 | 0.81855110 | | 1.813378 |
| | G/C | G/G | 6 | 1.00% | 83 | 3.06% | 7.185456 | 0.00734969 | p < 0.05 | 0.320433 |
| | | G/T | 146 | 24.33% | 643 | 23.67% | 0.084107 | 0.77180714 | | 1.036777 |
| | | T/T | 1 | 0.17% | 7 | 0.26% | 0.002328 | 0.96151780 | | 0.646077 |
| | G/G | G/G | | 0.00% | 8 | 0.29% | 0.759085 | 0.38361504 | | 0 |
| | | G/T | 6 | 1.00% | 8 | 0.29% | 4.260318 | 0.03901262 | p < 0.05 | 3.419192 |
| | | T/T | 1 | 0.17% | 13 | 0.48% | 0.516658 | 0.47227080 | | 0.347117 |
| G/G | C/C | G/G | 105 | 17.50% | 427 | 15.72% | 1.025599 | 0.31119482 | | 1.137109 |
| | | G/T | 2 | 0.33% | 21 | 0.77% | 0.815675 | 0.36644775 | | 0.429208 |
| | | T/T | | 0.00% | 3 | 0.11% | 0.004128 | 0.94876991 | | 0 |
| | G/C | G/G | | 0.00% | 20 | 0.74% | 3.301607 | 0.06921215 | | 0 |
| | | G/T | 2 | 0.33% | 62 | 2.28% | 8.863893 | 0.00290867 | p < 0.05 | 0.143165 |
| | | T/T | | 0.00% | 2 | 0.07% | 0.064399 | 0.79967293 | | 0 |
| | G/G | G/G | | 0.00% | 4 | 0.15% | 0.084565 | 0.77120372 | | 0 |
| | | G/T | | 0.00% | 1 | 0.04% | 0.687102 | 0.40715180 | | 0 |
| | | T/T | | 0.00% | 7 | 0.26% | 0.567662 | 0.45118949 | | 0 |
| Total | | | 600 | 100.00% | 2716 | 100.00% | | | | |

Summarization of haplotyping data using program HAPLO.STATS is presented in Tables 11-17. Comparisons between genotyping considering all possible combinations and haplotyping is showing unequivocally that combined genotypes are giving more appropriate data in diagnosting cancer risk in patients.

TABLE 11
Breast cancer

| loci | | | | Control | | |
|---|---|---|---|---|---|---|
| 48 | 119 | 432 | Case freq. | freq. | OR | p |
| G | G | C | 0.0079670 | 0.035358 | 0.2551 | 1.0991e−14 |
| G | T | G | 0.0062568 | 0.023563 | 0.30585 | 1.8132e−07 |
| C | T | G | 0.0021207 | 0.010802 | 0.23587 | 0.0015733 |
| G | G | G | 0.0054331 | 0.010623 | 0.71336 | 0.0016904 |
| C | G | G | 0.3958216 | 0.404570 | 1.0000 | 0.11453 |
| G | T | C | 0.2863496 | 0.253357 | 1.14240 | 0.0038601 |
| C | T | C | 0.0304894 | 0.018427 | 1.71023 | 0.0021405 |
| C | G | C | 0.2655619 | 0.243300 | 1.13315 | 0.0016186 |

12
Colon cancer

| loci | | | | Control | | |
|---|---|---|---|---|---|---|
| 48 | 119 | 432 | Case freq. | freq. | OR | p |
| G | T | G | 0.0051168 | 0.023563 | 0.28643 | 0.013235 |
| C | G | G | 0.3624949 | 0.404570 | 1.00000 | 0.032421 |
| C | G | C | 0.2358016 | 0.243300 | 1.05796 | 0.77006 |
| G | T | C | 0.2546966 | 0.253357 | 1.13388 | 0.98886 |
| G | G | G | 0.0139225 | 0.010623 | 1.29414 | 0.59231 |
| G | G | C | 0.0549158 | 0.035358 | 1.55316 | 0.037562 |
| C | T | C | 0.0346422 | 0.018427 | 1.94203 | 0.0003759 |
| C | T | G | 0.0384096 | 0.010802 | 2.71472 | 7.2671e−07 |

13
Kidney cancer

| loci | | | | Control | | |
|---|---|---|---|---|---|---|
| 48 | 119 | 432 | Case freq. | freq. | OR | p |
| G | G | C | 0.0076003 | 0.035358 | 0.21527 | 0.0053918 |
| G | T | G | 0.0030933 | 0.023563 | 0.12697 | 0.0291194 |
| G | G | G | 0 | 0.010623 | | 0.0523786 |
| C | T | G | 0 | 0.010802 | | 0.1049533 |
| C | T | C | 0.017551 | 0.018427 | 0.87234 | 0.6862900 |
| C | G | C | 0.23181 | 0.243300 | 0.84363 | 0.840498 |
| G | T | C | 0.28035 | 0.253357 | 0.94095 | 0.3564163 |
| C | G | G | 0.45959 | 0.404570 | 1.0000 | 0.0604614 |

14
Larynx cancer

| loci | | | | Control | | |
|---|---|---|---|---|---|---|
| 48 | 119 | 432 | Case freq. | freq. | OR | p |
| G | G | C | 0.0066386 | 0.035358 | 0.21701 | 0.00018354 |
| G | T | G | 0.0055984 | 0.023563 | 0.28602 | 0.01325223 |
| C | T | G | 0.0018561 | 0.010802 | 0.20576 | 0.07116657 |
| C | G | G | 0.3869071 | 0.404570 | 1.0000 | 0.20276932 |
| G | G | G | 0.0079046 | 0.010623 | 0.92231 | 0.43953288 |
| C | T | C | 0.0240185 | 0.018427 | 1.39157 | 0.41220093 |
| C | G | C | 0.2529406 | 0.243300 | 1.09852 | 0.36851527 |
| G | T | C | 0.3141360 | 0.253358 | 1.27393 | 0.00161746 |

15
Lung cancer

| loci | | | | Control | | |
|---|---|---|---|---|---|---|
| 48 | 119 | 432 | Case freq. | freq. | OR | p |
| G | G | C | 0.0091329 | 0.035358 | 0.27058 | 0.00051802 |
| G | G | G | 0 | 0.010623 | | 0.01732045 |
| C | T | G | 0 | 0.010802 | | 0.01735328 |
| G | T | G | 0.0079572 | 0.023563 | 0.34250 | 0.05386091 |
| C | T | C | 0.0091329 | 0.018427 | 0.51014 | 0.08247251 |
| C | G | C | 0.23296 | 0.243300 | 0.89289 | 0.82077489 |
| C | G | G | 0.43766 | 0.404570 | 1.0000 | 0.19548419 |
| G | T | C | 0.30315 | 0.253357 | 1.0689 | 0.01535562 |

16
Pancreas cancer

| loci | | | | Control | | |
|---|---|---|---|---|---|---|
| 48 | 119 | 432 | Case freq. | freq. | OR | p |
| G | G | C | 0.0156289 | 0.035358 | 0.47974 | 0.0275465 |
| C | T | G | 0 | 0.010802 | | 0.0470471 |
| G | G | G | 0 | 0.010623 | | 0.0547348 |
| G | T | G | 0.0057308 | 0.023563 | 0.27398 | 0.0683960 |
| C | G | C | 0.2169742 | 0.243300 | 0.87535 | 0.3635137 |
| C | T | C | 0.0156289 | 0.018427 | 0.89690 | 0.6654132 |
| C | G | G | 0.4171767 | 0.404570 | 1.0000 | 0.8747027 |
| G | T | C | 0.3288605 | 0.253357 | 1.2280 | 0.0012766 |

17
Prostate cancer

| loci | | | | Control | | |
|---|---|---|---|---|---|---|
| 48 | 119 | 432 | Case freq. | freq. | OR | p |
| G | G | C | 0.0097384 | 0.035358 | 0.30362 | 0.00003388 |
| G | T | G | 0.0031976 | 0.023563 | 0.15581 | 0.00060973 |
| G | G | G | 0.063762 | 0.010623 | 0.68268 | 0.059391 |
| C | T | G | 0.0056104 | 0.010802 | 0.58805 | 0.14350 |
| C | T | C | 0.0138374 | 0.018427 | 0.79436 | 0.27617 |
| C | G | C | 0.2249031 | 0.243300 | 0.92488 | 0.32647 |
| C | G | G | 0.4081491 | 0.404571 | 1.0000 | 0.86123 |
| G | T | C | 0.3281878 | 0.253357 | 1.24645 | 0.00000218 |

III. Results on Combined Genotypes Variants Including Alternative SNPs at Two of Three Codons—48,119 and 432.

The above results are presented in tables 18-38.

TABLE 18
Breast cancer

| CYP1B1 | | Cases | | Control | | | | | |
|---|---|---|---|---|---|---|---|---|---|
| variants | | | Freq. | | Freq. | Statistical analysis | | | |
| 432 | 48 | No. | (%) | No. | (%) | CHI2 | p-value | | OR |
| C/C | C/C | 157 | 8.49% | 203 | 7.17% | 2.552695 | 0.11010634 | | 1.20078 |
| | G/C | 274 | 14.82% | 418 | 14.77% | 1.28E−05 | 0.99714873 | | 1.003855 |
| | G/G | 189 | 10.22% | 240 | 8.48% | 3.864745 | 0.04931070 | p < 0.05 | 1.22869 |
| G/C | C/C | 497 | 26.88% | 612 | 21.63% | 16.78131 | 0.00004194 | p < 0.05 | 1.332263 |
| | G/C | 431 | 23.31% | 759 | 26.82% | 7.080768 | 0.00779156 | p < 0.05 | 0.829353 |
| | G/G | 15 | 0.81% | 31 | 1.10% | 0.658684 | 0.41702496 | | 0.73847 |
| G/G | C/C | 270 | 14.60% | 468 | 16.54% | 3.006724 | 0.08291973 | | 0.86301 |
| | G/C | 13 | 0.70% | 84 | 2.97% | 27.15903 | 0.00000019 | p < 0.05 | 0.231469 |
| | G/G | 3 | 0.08% | 15 | 0.53% | 3.04603 | 0.08093480 | | 0.304984 |
| Total | | 1849 | 100.00% | 2830 | 100.00% | | | | |

TABLE 19
Breast cancer

| CYP1B1 | | Cases | | Control | | | | | |
|---|---|---|---|---|---|---|---|---|---|
| variants | | | Freq. | | Freq. | Statistical analysis | | | |
| 432 | 119 | No. | (%) | No. | (%) | CHI2 | p-value | | OR |
| C/C | G/G | 134 | 6.96% | 252 | 8.55% | 3.83343 | 0.05024002 | | 0.799846 |
| | G/T | 307 | 15.95% | 436 | 14.80% | 1.10046 | 0.29416546 | | 1.092313 |
| | T/T | 199 | 10.34% | 212 | 7.20% | 14.46815 | 0.00014255 | p < 0.05 | 1.486877 |
| G/C | G/G | 479 | 24.88% | 673 | 22.84% | 2.567825 | 0.10905759 | | 1.118798 |
| | G/T | 508 | 26.39% | 766 | 26.00% | 0.071876 | 0.78862533 | | 1.020285 |
| | T/T | 4 | 0.21% | 27 | 0.92% | 8.160336 | 0.00428164 | p < 0.05 | 0.225114 |
| G/G | G/G | 277 | 14.39% | 477 | 16.19% | 2.753002 | 0.09707206 | | 0.870012 |
| | G/T | 16 | 0.83% | 89 | 3.02% | 25.44335 | 0.00000046 | p < 0.05 | 0.269051 |
| | T/T | 1 | 0.03% | 14 | 0.48% | 5.485606 | 0.01917368 | p < 0.05 | 0.108851 |
| Total | | 1925 | 100.00% | 2946 | 100.00% | | | | |

TABLE 20
Breast cancer

| CYP1B1 | | Cases | | Control | | | | | |
|---|---|---|---|---|---|---|---|---|---|
| variants | | | Freq. | | Freq. | Statistical analysis | | | |
| 48 | 119 | No. | (%) | No. | (%) | CHI2 | p-value | | OR |
| C/C | G/G | 843 | 44.63% | 1300 | 40.16% | 9.597386 | 0.00194855 | p < 0.05 | 1.200832 |
| | G/T | 99 | 5.24% | 126 | 3.89% | 4.851523 | 0.02762174 | p < 0.05 | 1.365563 |
| | T/T | 4 | 0.21% | 19 | 0.59% | 2.966599 | 0.08500039 | | 0.359402 |
| G/C | G/G | 16 | 0.85% | 183 | 5.65% | 72.56792 | 0.00000000 | p < 0.05 | 0.142561 |
| | G/T | 702 | 37.16% | 1242 | 38.37% | 0.687036 | 0.40717427 | | 0.949965 |
| | T/T | 16 | 0.85% | 24 | 0.74% | 0.062451 | 0.80266382 | | 1.14362 |
| G/G | G/G | 12 | 0.64% | 37 | 1.14% | 2.73441 | 0.09820779 | | 0.552924 |
| | G/T | 11 | 0.58% | 58 | 1.79% | 12.24506 | 0.00046649 | p < 0.05 | 0.32104 |
| | T/T | 186 | 9.8% | 248 | 7.66% | 7.069853 | 0.00783917 | p < 0.05 | 1.316353 |
| Total | | 1889 | 100.00% | 3237 | 100.00% | | | | |

TABLE 21
Colon cancer

| CYP1B1 | | | | | | | | |
|---|---|---|---|---|---|---|---|---|
| variants | | Cases | | Control | | Statistical analysis | | |
| 432 | 48 | No. | Freq. (%) | No. | Freq. (%) | CHI2 | p-value | OR |
| C/C | C/C | 30 | 8.40% | 203 | 7.17% | 0.538095 | 0.46322332 | 1.187237 |
| | G/C | 57 | 15.97% | 418 | 14.77% | 0.269486 | 0.60367666 | 1.096364 |
| | G/G | 37 | 10.36% | 240 | 8.48% | 1.189798 | 0.27537051 | 1.247786 |
| G/C | C/C | 73 | 20.45% | 612 | 21.63% | 0.195284 | 0.65855489 | 0.931568 |
| | G/C | 89 | 24.93% | 759 | 26.82% | 0.487027 | 0.48525671 | 0.906136 |
| | G/G | 4 | 1.12% | 31 | 1.10% | 0.051382 | 0.82067656 | 1.02312 |
| G/G | C/C | 61 | 17.09% | 468 | 16.54% | 0.03519 | 0.85119850 | 1.040093 |
| | G/C | 4 | 1.12% | 84 | 2.97% | 3.372245 | 0.06630335 | 0.37043 |
| | G/G | 2 | 0.28% | 15 | 0.53% | 0.097183 | 0.75523730 | 1.057277 |
| Total | | 357 | 100.00% | 2830 | 100.00% | | | |

TABLE 22
Colon cancer

| CYP1B1 | | | | | | | | | |
|---|---|---|---|---|---|---|---|---|---|
| variants | | Cases | | Control | | Statistical analysis | | | |
| 432 | 119 | No. | Freq. (%) | No. | Freq. (%) | CHI2 | p-value | | OR |
| C/C | G/G | 50 | 11.04% | 252 | 8.55% | 2.692536 | 0.10081930 | | 1.326362 |
| | G/T | 70 | 15.45% | 436 | 14.80% | 0.085559 | 0.76990043 | | 1.052171 |
| | T/T | 44 | 9.71% | 212 | 7.20% | 3.218875 | 0.07279367 | | 1.387369 |
| G/C | G/G | 83 | 18.32% | 673 | 22.84% | 4.384898 | 0.03625866 | p < 0.05 | 0.757636 |
| | G/T | 122 | 26.93% | 766 | 26.00% | 0.131131 | 0.71726257 | | 1.048962 |
| | T/T | 2 | 0.44% | 27 | 0.92% | 0.560964 | 0.45387226 | | 0.479428 |
| G/G | G/G | 68 | 15.01% | 477 | 16.19% | 0.323399 | 0.56957206 | | 0.91422 |
| | G/T | 6 | 1.32% | 89 | 3.02% | 3.558542 | 0.05923974 | | 0.430888 |
| | T/T | 8 | 0.88% | 14 | 0.48% | 8.264443 | 0.00404291 | p < 0.05 | 3.765008 |
| | | 453 | 100.00% | 2946 | 100.00% | | | | |

TABLE 23
Colon cancer

| CYP1B1 | | | | | | | | | |
|---|---|---|---|---|---|---|---|---|---|
| variants | | Cases | | Control | | Statistical analysis | | | |
| 48 | 119 | No. | Freq. (%) | No. | Freq. (%) | CHI2 | p-value | | OR |
| C/C | G/G | 134 | 35.83% | 1300 | 40.16% | 2.449813 | 0.11753890 | | 0.831917 |
| | G/T | 27 | 7.22% | 126 | 3.89% | 8.343146 | 0.00387145 | p < 0.05 | 1.921161 |
| | T/T | 10 | 2.67% | 19 | 0.59% | 15.8012 | 0.00007036 | p < 0.05 | 4.652979 |
| G/C | G/G | 21 | 5.61% | 183 | 5.65% | 0.007712 | 0.93002314 | | 0.992802 |
| | G/T | 136 | 36.36% | 1242 | 38.37% | 0.489441 | 0.48417658 | | 0.917874 |
| | T/T | 2 | 0.53% | 24 | 0.74% | 0.015524 | 0.90084513 | | 0.719758 |
| G/G | G/G | 13 | 3.48% | 37 | 1.14% | 11.70879 | 0.00062206 | p < 0.05 | 3.114472 |
| | G/T | 3 | 0.80% | 58 | 1.79% | 1.426154 | 0.23239322 | | 0.44321 |
| | T/T | 28 | 3.74% | 248 | 7.66% | 0.000312 | 0.98589792 | | 0.97534 |
| total | | 374 | 100.00% | 3237 | 100.00% | | | | |

TABLE 24
Kidney cancer

| CYP1B1 | | | | | | | | | |
|---|---|---|---|---|---|---|---|---|---|
| variants | | Cases | | Control | | Statistical analysis | | | |
| 432 | 48 | No. | Freq. (%) | No. | Freq. (%) | CHI2 | p-value | | OR |
| C/C | C/C | 14 | 6.83% | 203 | 7.17% | 0.001951 | 0.96477256 | | 0.948547 |
| | G/C | 28 | 13.66% | 418 | 14.77% | 0.110226 | 0.73988651 | | 0.912821 |
| | G/G | 22 | 10.73% | 240 | 8.48% | 0.959328 | 0.32735632 | | 1.297359 |
| G/C | C/C | 46 | 22.44% | 612 | 21.63% | 0.034304 | 0.85306220 | | 1.048506 |
| | G/C | 45 | 21.95% | 759 | 26.82% | 2.083433 | 0.14890506 | | 0.767416 |
| | G/G | 1 | 0.49% | 31 | 1.10% | 0.219394 | 0.63950226 | | 0.4426 |
| G/G | C/C | 49 | 23.90% | 468 | 16.54% | 6.825431 | 0.00898689 | p < 0.05 | 1.585278 |
| | G/C | | 0.00% | 84 | 2.97% | 5.203643 | 0.02253960 | p < 0.05 | 0 |
| | G/G | | 0.00% | 15 | 0.53% | 0.280141 | 0.59660897 | | 0 |
| Total | | 205 | 100.00% | 2830 | 100.00% | | | | |

TABLE 25
Kidney cancer

| CYP1B1 | | | | | | | | | |
|---|---|---|---|---|---|---|---|---|---|
| variants | | Cases | | Control | | Statistical analysis | | | |
| 432 | 119 | No. | Freq. (%) | No. | Freq. (%) | CHI2 | p-value | | OR |
| C/C | G/G | 12 | 5.77% | 252 | 8.55% | 1.618074 | 0.20336062 | | 0.654519 |
| | G/T | 33 | 15.87% | 436 | 14.80% | 0.100273 | 0.75150196 | | 1.085583 |
| | T/T | 20 | 9.62% | 212 | 7.20% | 1.332393 | 0.24838004 | | 1.371939 |
| G/C | G/G | 42 | 20.19% | 673 | 22.84% | 0.635629 | 0.42529789 | | 0.854527 |
| | G/T | 52 | 25.00% | 766 | 26.00% | 0.055986 | 0.81295673 | | 0.948651 |
| | T/T | 1 | 0.48% | 27 | 0.92% | 0.070252 | 0.79097017 | | 0.522276 |
| G/G | G/G | 48 | 23.08% | 477 | 16.19% | 6.151608 | 0.01312927 | p < 0.05 | 1.55283 |
| | G/T | | 0.00% | 89 | 3.02% | 5.411473 | 0.02000483 | p < 0.05 | 0 |
| | T/T | | 0.00% | 14 | 0.48% | 0.208675 | 0.64780790 | | 0 |
| Total | | 208 | 100.00% | 2946 | 100.00% | | | | |

TABLE 26
Kidney cancer

| CYP1B1 | | | | | | | | | |
|---|---|---|---|---|---|---|---|---|---|
| variants | | Cases | | Control | | Statistical analysis | | CYP1B1 variants | |
| 48 | 119 | No. | Freq. (%) | No. | Freq. (%) | CHI2 | p-value | | OR |
| C/C | G/G | 102 | 49.04% | 1300 | 40.16% | 6.019961 | 0.01414496 | p < 0.05 | 1.433774 |
| | G/T | 7 | 3.37% | 126 | 3.89% | 0.038751 | 0.84394320 | | 0.859867 |
| | T/T | | 0.00% | 19 | 0.59% | 0.390712 | 0.53192518 | | 0 |
| G/C | G/G | | 0.00% | 183 | 5.65% | 11.32019 | 0.00076669 | p < 0.05 | 0 |
| | G/T | 75 | 36.06% | 1242 | 38.37% | 0.349626 | 0.55432487 | | 0.905797 |
| | T/T | | 0.00% | 24 | 0.74% | 0.666165 | 0.41439200 | | 0 |
| G/G | G/G | 1 | 0.48% | 37 | 1.14% | 0.29595 | 0.58643261 | | 0.417809 |
| | G/T | 1 | 0.48% | 58 | 1.79% | 1.29274 | 0.25554374 | | 0.264784 |
| | T/T | 22 | 10.58% | 248 | 7.66% | 1.914016 | 0.16651789 | | 1.425555 |
| Total | | 208 | 100.00% | 3237 | 100.00% | | | | |

TABLE 27
Larynx cancer

| CYP1B1 | | | | | | | | | |
|---|---|---|---|---|---|---|---|---|---|
| variants | | Cases | | Control | | Statistical analysis | | | |
| 432 | 48 | No. | Freq. (%) | No. | Freq. (%) | CHI2 | p-value | | OR |
| C/C | C/C | 23 | 6.52% | 203 | 7.17% | 0.118124 | 0.73107810 | | 0.901941 |
| | G/C | 69 | 19.55% | 418 | 14.77% | 5.162856 | 0.02307493 | p < 0.05 | 1.401948 |
| | G/G | 36 | 10.20% | 240 | 8.48% | 0.962531 | 0.32654992 | | 1.225552 |
| G/C | C/C | 79 | 22.38% | 612 | 21.63% | 0.065341 | 0.79824470 | | 1.044929 |
| | G/C | 85 | 24.08% | 759 | 26.82% | 1.073134 | 0.30023885 | | 0.865411 |
| | G/G | 2 | 0.57% | 31 | 1.10% | 0.417693 | 0.51809066 | | 0.514475 |
| G/G | C/C | 55 | 15.58% | 468 | 16.54% | 0.145208 | 0.70315732 | | 0.931495 |
| | G/C | 2 | 0.57% | 84 | 2.97% | 6.002764 | 0.01428349 | p < 0.05 | 0.186271 |
| | G/G | 2 | 0.28% | 15 | 0.53% | 0.089053 | 0.76538396 | | 1.069326 |
| Total | | 353 | 100.00% | 2830 | 100.00% | | | | |

TABLE 28
Larynx cancer

| CYP1B1 | | | | | | | | | |
|---|---|---|---|---|---|---|---|---|---|
| variants | | Cases | | Control | | Statistical analysis | | | |
| 432 | 119 | No. | Freq. (%) | No. | Freq. (%) | CHI2 | p-value | | OR |
| C/C | G/G | 24 | 6.58% | 252 | 8.55% | 1.415096 | 0.23421240 | | 0.752409 |
| | G/T | 60 | 16.44% | 436 | 14.80% | 0.562062 | 0.45343067 | | 1.132501 |
| | T/T | 45 | 12.33% | 212 | 7.20% | 11.24343 | 0.00079905 | p < 0.05 | 1.813532 |
| G/C | G/G | 78 | 21.37% | 673 | 22.84% | 0.323015 | 0.56980161 | | 0.917904 |
| | G/T | 95 | 26.03% | 766 | 26.00% | 0.002762 | 0.95808852 | | 1.001354 |
| | T/T | 1 | 0.27% | 27 | 0.92% | 0.92449 | 0.33629886 | | 0.297009 |
| G/G | G/G | 59 | 16.16% | 477 | 16.19% | 0.003854 | 0.95049631 | | 0.998006 |
| | G/T | 3 | 0.82% | 89 | 3.02% | 5.028454 | 0.02493415 | p < 0.05 | 0.266031 |
| | T/T | | 0.00% | 14 | 0.48% | 0.79608 | 0.37226783 | | 0 |
| Total | | 365 | 100.00% | 2946 | 100.00% | | | | |

TABLE 29
Larynx cancer

| CYP1B1 | | | | | | | | | |
|---|---|---|---|---|---|---|---|---|---|
| variants | | Cases | | Control | | Statistical analysis | | | |
| 48 | 119 | No. | Freq. (%) | No. | Freq. (%) | CHI2 | p-value | | OR |
| C/C | G/G | 177 | 42.86% | 1300 | 40.16% | 0.996372 | 0.31819002 | | 1.1175 |
| | G/T | 9 | 2.18% | 126 | 3.89% | 2.556714 | 0.10982665 | | 0.550035 |
| | T/T | 1 | 0.24% | 19 | 0.59% | 0.291685 | 0.58914285 | | 0.411088 |
| G/C | G/G | 3 | 0.73% | 183 | 5.65% | 17.38011 | 0.00003060 | p < 0.05 | 0.122111 |
| | G/T | 168 | 40.68% | 1242 | 38.37% | 0.729249 | 0.39312678 | | 1.101449 |
| | T/T | 9 | 2.18% | 24 | 0.74% | 6.922078 | 0.00851380 | p < 0.05 | 2.982364 |
| G/G | G/G | 3 | 0.73% | 37 | 1.14% | 0.265177 | 0.60658529 | | 0.632828 |
| | G/T | 1 | 0.24% | 58 | 1.79% | 4.599266 | 0.03198564 | p < 0.05 | 0.133035 |
| | T/T | 42 | 5.08% | 248 | 7.66% | 2.816556 | 0.09329643 | | 1.364425 |
| Total | | 413 | 100.00% | 3237 | 100.00% | | | | |

TABLE 30
Lung cancer

| CYP1B1 | | | | | | | | | |
|---|---|---|---|---|---|---|---|---|---|
| variants | | Cases | | Control | | Statistical analysis | | | |
| 432 | 48 | No. | Freq. (%) | No. | Freq. (%) | CHI2 | p-value | | OR |
| C/C | C/C | 20 | 6.04% | 203 | 7.17% | 0.418354 | 0.51775949 | | 0.832211 |
| | G/C | 44 | 13.29% | 418 | 14.77% | 0.406602 | 0.52369949 | | 0.884651 |
| | G/G | 33 | 9.97% | 240 | 8.48% | 0.654869 | 0.41837720 | | 1.19505 |
| G/C | C/C | 75 | 22.66% | 612 | 21.63% | 0.130183 | 0.71824198 | | 1.061772 |
| | G/C | 95 | 28.70% | 759 | 26.82% | 0.440697 | 0.50678608 | | 1.098373 |
| | G/G | 3 | 0.91% | 31 | 1.10% | 0.001152 | 0.97292594 | | 0.825826 |
| G/G | C/C | 60 | 18.13% | 468 | 16.54% | 0.43011 | 0.51193511 | | 1.117419 |
| | G/C | 1 | 0.30% | 84 | 2.97% | 7.063098 | 0.00786878 | p < 0.05 | 0.099062 |
| | G/G | | 0.00% | 15 | 0.53% | 0.819123 | 0.36543670 | | 0 |
| Total | | 331 | 100.00% | 2830 | 100.00% | | | | |

TABLE 31
Lung cancer

| CYP1B1 | | Cases | | Control | | | | | |
|---|---|---|---|---|---|---|---|---|---|
| variants | | | Freq. | | Freq. | Statistical analysis | | | |
| 432 | 119 | No. | (%) | No. | (%) | CHI2 | p-value | | OR |
| C/C | G/G | 24 | 5.85% | 252 | 8.55% | 3.128456 | 0.07693660 | | 0.664693 |
| | G/T | 51 | 12.44% | 436 | 14.80% | 1.432127 | 0.23141782 | | 0.81783 |
| | T/T | 42 | 10.24% | 212 | 7.20% | 4.352964 | 0.03694461 | p < 0.05 | 1.471852 |
| G/C | G/G | 92 | 22.44% | 673 | 22.84% | 0.014534 | 0.90404156 | | 0.977114 |
| | G/T | 121 | 29.51% | 766 | 26.00% | 2.10449 | 0.14686739 | | 1.191558 |
| | T/T | 3 | 0.73% | 27 | 0.92% | 0.008546 | 0.92634326 | | 0.796888 |
| G/G | G/G | 76 | 18.54% | 477 | 16.19% | 1.272877 | 0.25922787 | | 1.177795 |
| | G/T | 1 | 0.24% | 89 | 3.02% | 9.598465 | 0.00194740 | p < 0.05 | 0.078487 |
| | T/T | | 0.00% | 14 | 0.48% | 0.97983 | 0.32224079 | | 0 |
| Total | | 410 | 100.00% | 2946 | 100.00% | | | | |

TABLE 32
Lung cancer

| CYP1B1 | | Cases | | Control | | | | | |
|---|---|---|---|---|---|---|---|---|---|
| variants | | | Freq. | | Freq. | Statistical analysis | | | |
| 48 | 119 | No. | (%) | No. | (%) | CHI2 | p-value | | OR |
| C/C | G/G | 155 | 45.86% | 1300 | 40.16% | 3.883383 | 0.04876621 | p < 0.05 | 1.262022 |
| | G/T | 4 | 1.18% | 126 | 3.89% | 5.659948 | 0.01735664 | p < 0.05 | 0.295694 |
| | T/T | | 0.00% | 19 | 0.59% | 1.038741 | 0.30811487 | | 0 |
| G/C | G/G | 6 | 1.78% | 183 | 5.65% | 8.434735 | 0.00368120 | p < 0.05 | 0.3016 |
| | G/T | 135 | 39.94% | 1242 | 38.37% | 0.256416 | 0.61259329 | | 1.068216 |
| | T/T | 2 | 0.59% | 24 | 0.74% | 0.000791 | 0.97755639 | | 0.796875 |
| G/G | G/G | | 0.00% | 37 | 1.14% | 2.867643 | 0.09037715 | | 0 |
| | G/T | | 0.00% | 58 | 1.79% | 5.08465 | 0.02413855 | p < 0.05 | 0 |
| | T/T | 36 | 10.65% | 248 | 7.66% | 3.342439 | 0.06751450 | | 1.436712 |
| Total | | 338 | 100.00% | 3237 | 100.00% | | | | |

TABLE 33
Pancreas cancer

| CYP1B1 | | Cases | | Control | | | | | |
|---|---|---|---|---|---|---|---|---|---|
| variants | | | Freq. | | Freq. | Statistical analysis | | | |
| 432 | 48 | No. | (%) | No. | (%) | CHI2 | p-value | | OR |
| C/C | C/C | 16 | 7.05% | 203 | 7.17% | 0.004052 | 0.94924333 | | 0.981299 |
| | G/C | 34 | 14.98% | 418 | 14.77% | 0.000153 | 0.99013455 | | 1.016536 |
| | G/G | 26 | 11.45% | 240 | 8.48% | 1.979062 | 0.15948935 | | 1.395937 |
| G/C | C/C | 39 | 17.18% | 612 | 21.63% | 2.218983 | 0.13632285 | | 0.751825 |
| | G/C | 70 | 30.84% | 759 | 26.82% | 1.518648 | 0.21782432 | | 1.216569 |
| | G/G | 1 | 0.44% | 31 | 1.10% | 0.352688 | 0.55259526 | | 0.399515 |
| G/G | C/C | 40 | 17.62% | 468 | 16.54% | 0.108575 | 0.74177270 | | 1.079574 |
| | G/C | 1 | 0.44% | 84 | 2.97% | 4.075785 | 0.04350206 | p < 0.05 | 0.144648 |
| | G/G | | 0.00% | 15 | 0.53% | 0.367223 | 0.54452190 | | 0 |
| Total | | 227 | 100.00% | 2830 | 100.00% | | | | |

TABLE 34
Pancreas cancer

| CYP1B1 | | Cases | | Control | | | | | |
|---|---|---|---|---|---|---|---|---|---|
| variants | | | Freq. | | Freq. | Statistical analysis | | | |
| 432 | 119 | No. | (%) | No. | (%) | CHI2 | p-value | | OR |
| C/C | G/G | 17 | 7.14% | 252 | 8.55% | 0.399152 | 0.52752768 | | 0.822344 |
| | G/T | 33 | 13.87% | 436 | 14.80% | 0.087667 | 0.76716493 | | 0.926717 |
| | T/T | 29 | 12.18% | 212 | 7.20% | 7.136493 | 0.00755307 | p < 0.05 | 1.789429 |
| G/C | G/G | 44 | 18.49% | 673 | 22.84% | 2.152842 | 0.14230630 | | 0.766012 |
| | G/T | 73 | 30.67% | 766 | 26.00% | 2.240772 | 0.13441390 | | 1.259119 |
| | T/T | 1 | 0.42% | 27 | 0.92% | 0.183178 | 0.66865658 | | 0.456165 |
| G/G | G/G | 40 | 16.81% | 477 | 16.19% | 0.024402 | 0.87586631 | | 1.045677 |
| | G/T | 1 | 0.42% | 89 | 3.02% | 4.517685 | 0.03354620 | p < 0.05 | 0.135448 |
| | T/T | | 0.00% | 14 | 0.48% | 0.309795 | 0.57780594 | | 0 |
| Total | | 238 | 100.00% | 2946 | 100.00% | | | | |

TABLE 35
Pancreas cancer

| CYP1B1 | | Cases | | Control | | | | |
|---|---|---|---|---|---|---|---|---|
| variants | | | Freq. | | Freq. | Statistical analysis | | |
| 48 | 119 | No. | (%) | No. | (%) | CHI2 | p-value | OR |
| C/C | G/G | 91 | 40.09% | 1300 | 40.16% | 0.00235 | 0.96133388 | 0.996985 |
| | G/T | 4 | 1.76% | 126 | 3.89% | 2.10816 | 0.14651542 | 0.442878 |
| | T/T | | 0.00% | 19 | 0.59% | 0.47978 | 0.48852180 | 0 |
| G/C | G/G | 6 | 2.64% | 183 | 5.65% | 3.165504 | 0.07520923 | 0.453082 |
| | G/T | 96 | 42.29% | 1242 | 38.37% | 1.215849 | 0.27017699 | 1.177121 |
| | T/T | 3 | 1.32% | 24 | 0.74% | 0.325425 | 0.56836578 | 1.792969 |
| G/G | G/G | | 0.00% | 37 | 1.14% | 1.652549 | 0.19861240 | 0 |
| | G/T | 1 | 0.44% | 58 | 1.79% | 1.576704 | 0.20923627 | 0.242524 |
| | T/T | 26 | 11.45% | 248 | 7.66% | 3.683663 | 0.05494803 | 1.559019 |
| Total | | 227 | 100.00% | 3237 | 100.00% | | | |

TABLE 36
Prostate cancer

| CYP1B1 | | Cases | | Control | | | | | |
|---|---|---|---|---|---|---|---|---|---|
| variants | | | Freq. | | Freq. | Statistical analysis | | | |
| 432 | 48 | No. | (%) | No. | (%) | CHI2 | p-value | | OR |
| C/C | C/C | 38 | 6.33% | 203 | 7.17% | 0.413643 | 0.52012598 | | 0.875007 |
| | G/C | 78 | 13.00% | 418 | 14.77% | 1.115238 | 0.29094636 | | 0.862234 |
| | G/G | 85 | 14.17% | 240 | 8.48% | 18.00316 | 0.00002205 | p < 0.05 | 1.781149 |
| G/C | C/C | 130 | 21.67% | 612 | 21.63% | 0.001043 | 0.97423132 | | 1.002434 |
| | G/C | 153 | 25.50% | 759 | 26.82% | 0.376736 | 0.53935545 | | 0.933947 |
| | G/G | 7 | 1.17% | 31 | 1.10% | 0.003997 | 0.94959185 | | 1.065822 |
| G/G | C/C | 107 | 17.83% | 468 | 16.54% | 0.506828 | 0.47651523 | | 1.095395 |
| | G/C | 2 | 0.33% | 84 | 2.97% | 13.0027 | 0.00031104 | p < 0.05 | 0.109333 |
| | G/G | | 0.00% | 15 | 0.53% | 2.092828 | 0.14799194 | | 0 |
| Total | | 600 | 100.00% | 2830 | 100.00% | | | | |

TABLE 37
Prostate cancer

| CYP1B1 | | Cases | | Control | | | | | |
|---|---|---|---|---|---|---|---|---|---|
| variants | | | Freq. | | Freq. | | | | |
| 432 | 119 | No. | (%) | No. | (%) | CHI2 | p-value | | OR |
| C/C | G/G | 46 | 6.74% | 252 | 8.55% | 2.170118 | 0.14071544 | | 0.773211 |
| | G/T | 91 | 13.34% | 436 | 14.80% | 0.832695 | 0.36149451 | | 0.886423 |
| | T/T | 89 | 13.05% | 212 | 7.20% | 24.17787 | 0.00000088 | p < 0.05 | 1.935521 |
| G/C | G/G | 142 | 20.82% | 673 | 22.84% | 1.188177 | 0.27569775 | | 0.888135 |
| | G/T | 185 | 27.13% | 766 | 26.00% | 0.306393 | 0.57990176 | | 1.059359 |
| | T/T | 4 | 0.59% | 27 | 0.92% | 0.375596 | 0.53996965 | | 0.637824 |
| G/G | G/G | 119 | 17.45% | 477 | 16.19% | 0.549303 | 0.45860250 | | 1.09406 |
| | G/T | 6 | 0.88% | 89 | 3.02% | 9.135788 | 0.00250658 | p < 0.05 | 0.284921 |
| | T/T | | 0.00% | 14 | 0.48% | 2.134727 | 0.14399616 | | 0 |
| Total | | 682 | 100.00% | 2946 | 100.00% | | | | |

TABLE 38
Prostate cancer

| CYP1B1 | | Cases | | Control | | | | | |
|---|---|---|---|---|---|---|---|---|---|
| variants | | | Freq. | | Freq. | Statistical analysis | | | |
| 48 | 119 | No. | (%) | No. | (%) | CHI2 | p-value | | OR |
| C/C | G/G | 271 | 42.81% | 1300 | 40.16% | 1.435421 | 0.23088186 | | 1.115442 |
| | G/T | 15 | 2.37% | 126 | 3.89% | 3.077083 | 0.07940286 | | 0.599283 |
| | T/T | 2 | 0.32% | 19 | 0.59% | 0.305868 | 0.58022670 | | 0.536825 |
| G/C | G/G | 10 | 1.58% | 183 | 5.65% | 17.69258 | 0.00002596 | p < 0.05 | 0.267874 |
| | G/T | 231 | 36.49% | 1242 | 38.37% | 0.712811 | 0.39851205 | | 0.923012 |
| | T/T | 6 | 0.95% | 24 | 0.74% | 0.086353 | 0.76886579 | | 1.2811 |
| G/G | G/G | 1 | 0.16% | 37 | 1.14% | 4.319501 | 0.03767797 | p < 0.05 | 0.136846 |
| | G/T | 11 | 1.74% | 58 | 1.79% | 0.004937 | 0.94398273 | | 0.969315 |
| | T/T | 86 | 13.59% | 248 | 7.66% | 22.823 | 0.00000178 | p < 0.05 | 1.894896 |
| Total | | 633 | 100.00% | 3237 | 100.00% | | | | |

As expected sensitivity of studied variants (based on 2 SNPS) in detection of increased risk of cancer is higher than for variants based on 3 SNPs. By identification of these variants it is possible to detect the following proportion of patients affected by cancers:

| | Combined genotypes: | % of cancers: |
|---|---|---|
| Breast | TT¹¹⁹ CC⁴³² | 61.45 |
| | GG⁴⁸ CC⁴³² | |
| | CC⁴⁸ GC⁴³² | |
| | CC⁴⁸ GG¹¹⁹ | |
| | CC⁴⁸ GT¹¹⁹ | |
| | GG⁴⁸ TT¹¹⁹ | |
| Colon | TT¹¹⁹ GG⁴³² | 12.0 |
| | CC⁴⁸ GT¹¹⁹ | |
| | CC⁴⁸ TT¹¹⁹ | |
| | GG⁴⁸ GG¹¹⁹ | |
| Kidney | CC⁴⁸ GG⁴³² | 49.0 |
| | CC⁴⁸ GG¹¹⁹ | |
| | GG¹¹⁹ GG⁴³² | |
| Larynx | GC⁴⁸ TT¹¹⁹ | 28.9 |
| | TT¹¹⁹ CC⁴³² | |
| | GC⁴⁸ CC⁴³² | |
| Lung | CC⁴⁸ GG¹¹⁹ | 56.2 |
| | TT¹¹⁹ CC⁴³² | |
| Pancreas | TT¹¹⁹ CC⁴³² | 12.2 |
| Prostate | GG⁴⁸ CC⁴³² | 15.4 |
| | GG⁴⁸ TT¹¹⁹ | |
| | TT¹¹⁹ CC⁴³² | |

The herein invention, is showing for the first time that constitutional alterations of CYP1B1 355 T/T or other variants (presented in Table A and B) are the markers of significantly increased or decreased susceptibility to cancers of various sites, especially to tumor types described above. Suggested DNA testing is allowing identification of groups of individuals who should be covered by special programs of surveillance and prevention.

### REFERENCES

1. Bailey, L. R., Roodi, N., Dupont, W. D. and Parl, F. F. (1998) Association of cytochrome P450 1B1 (CYP1B1) polymorphism with steriod receptor status in breast cancer. Cancer Res., 58, 5038-5041.
2. Chang, B. L., Zheng S. L., Isaacs S. D., Turner A. R., Hawkins G. A., Wiley K. E., Bleecker E. R., (2003) Polymorphisms in the CYP1B1 gene are associated with increased risk of prostate cancer. British Journal of Cancer, 89,1524-1529
3. Chakravarti, D., Mailander, P. C., Li, K. M., Higginbotham, S., Zhang, H. L., Gross, M., Cavalieri E. L., Rogan, E. G., (2001) Evidence that a burst of DNA depurination in SENCAR mouse skin induces error-prone repair and forms mutations in the H-ras gene, Oncogene, 20, 7945-7953
4. Cicek, M. S., Liu, X., Casey, G., Witte, J. S., (2005) Role of androgen metabolism genes CYP1B1, PSA/KLK3, and CYP11alpha in prostate cancer risk and aggressiveness. Cancer Epidemiol Biomarkers Prev., 14(9),2173-7
5. Fritsche, E., Bruning, T., Jonkmanns, C., (1999) Detection of cytochrome P4501B1 Bfr I polymorphism: genotype distribution in healthy German individuals and in patients with colorectal carcinoma. Pharmacogenetics, 9,405-408
6. Goodman, M. T., McDuffie, K., Kolonel, L. N., Terada K., (2001) Case-control study of ovarian cancer and polymorphism in genes involved in catecholestrogen formation and metabolism
7. Gotoh, O., Tagashira Y., Lizuka T., Fujii-Kuriyama Y., (1983) Structural characteristic of cytochrome P450 ; possible location of the heme-binding cysteine in determined amino acid sequnece. J. Biochem, 93,807-817
8. Han, X., Liehr, J. G., (1994) DNA single-strand breaks in kidneys of Syrian hamster treated with steroidal estrogens:hormone-induced free radical damage preceding renal malignancy. Carcinogenesis, 15,997-1000
9. Hanna, I. H., Dawling, S., Roodi, N., Guengerich, F. P. and Parl, F. F. (2000) Cytochrome P450 1B1 (CYP1B1) pharmoacogenetics: association of polymorphisms with functional differences in estrogen hydroxylation activity. Cancer Res., 60,3440-3444
10. Hayes, C. L., Spink, D. C., Spink, B. C., Cao, J. Q., Walker, N. J., Sutter T. R., (1996) 17β-Estradiol hydroxylation catalyzed by human cytochrome P450 1B1, Proc. Natl. Acad. Sci. USA, 93,9776-9781
11. Jaworowska, E., Masojć, B., Tarnowska, C., Brzosko, M., Fliciński, J., Serrano-Fernandez, P., Matyjasik, J., Amernik, K., Scott, R., J., Lubinski, J., (2006) Association between early-onset breast and laryngeal cancers. Breast Cancer Res. Treat., 97(2),215-9
12. Rylander-Rudqvist, T., Wedren, S., Fredrik, G., Humphreys, K., Ahlberg, S., (2003) Cytochrome P450 1B1 gene polymorphisms and post menopausal breast cancer risk. Carcinogenesis, 24,1533-1539
13. Sasaki, M., Tanaka, Y., Okino S. T., Nomoto, M., Yonezawa, S., (2004) Polymorphisms of the CYP1B1 gene as risk factor for human renal cell cancer. Clinical Cancer Research, 10,2015-2019
14. Shimada, T., Hayes C. L., Yamazaki, H., Amin, S., Hecht, S. S., et al. (1996) Activation of chemically diverse procarcinogens by human cytochrome P-450 1B1. Cancer Res, 56,2979-2984
15. Shimada. T., Watanabe, J., Kawajiri, K., Sutter, R. T., Guengerich P. F., Gillam, M. J., Inoue, K., (1999) Catalytic properties of polymorphic human cytochrome P4501B1 variants. Carcinogenesis, 20/8,1607-1613
16. Spink, D. C., Eugster, H., Lincoln II, D. W., Schuetz, J. D., Schuetz, E. G., Johnson, J. A., Kaminsky, L. S., Gierthy, J. F., (1992)17β-Estradiol hydroxylation catalyzed by human cytochrome P450 1A1: a comparison of the activities induced by 2,3,7,8,-tetrachlorodibenzo-p-dioxin in MCF-7 cells with those from heterologous expression of the cDNA. Arch. Biochem. Biophys., 293,342-348
17. Spink, D. C., Hayes, C. L., Young, N. R., Christou, M., Sutter, T. R., Jefcoate, C. R., Gierthy, J. F., (1994) The effects of 2,3,7,8-tetrachlorodibenzo-p-dioxin on estrogen metabolism in MCF-7 breast cancer cells: evidence for induction of a novel 17β-estradiol 4-hydroxylase. J. Steroid Biochem. Mol. Biol., 51,251-258
18. Spink, D. C., Spink, B. C., Cao, J. Q., Induction of cytochrome P450 1B1 and Catechol Estrogen Metbolism in ACHN Human Renal Adenocarcinoma Cells. (1997) J. Steriod. Biochem. Molec. Biol. 2/3,223-232
19. Tanaka, Y., Sasaki M., Kaneuchi, M., Shiina, H., Igawa, M., Dahiya R., (2002) Polymorphisms of the CYP1B1 gene have higher risk for prostate cancer. BBRC, 296,820-826
20. Watanabe, J., Shimada, T., Gillam E., Ikuto T., Suemasu K., Higashi, Y., (2000) Association of CYP1B1 genetic polymorphism with incidence to breast and lung cancer. Pharmacogenetic, 10,25-33
21. Rylander-Rudqvist, T., Wedren, S., Jonasdottir G., (2004) Cytochrome P450 1B1 gene polymorphism and postmenopausal endometrial cancer risk. Can.Epidem.Biomarkers. Prev. 13(9)-1515-20.

1. A method for determining predisposition of a human subject to cancer comprising determining whether the CYP1B1 gene of the human subject has the genotype 355 T/T, 355 G/T, 355 G/G, 142 G/C, 142 C/C, 142 G/G, 4329 C/C, 4329 G/C or 4329 G/C, wherein the presence of the genotype is indicative of predisposition of the human subject to cancer.

2. The method of claim 1 for determining predisposition of a human subject to cancer comprising determining whether the CYP1B1 gene of the human subject has at least two codon variations selected from the group consisting of A119S alteration, R48G alteration and L432V alteration, wherein the presence of the codon variations is indicative of predisposition of the human subject to cancer.

3. The method of claim 2, wherein the A119S alteration results from the genotype 355 T/T, 355 G/T or 355 G/G, wherein the R48G alteration results from the genotype 142 G/C, 142 C/C or 142 G/G; and wherein the L432V alteration results from the genotype 4329 C/C, 4329 G/C or 4329 G/G.

4. The method of claim 3, wherein the identified genotype of the CYP1B1 gene indicative of significantly increased predisposition to cancer is the following combined genotype:
CYP1B1 gene combined genotype 4329 C/C, 142 G/G, 355 T/T being indicative of significantly increased predisposition to at least one of the following cancers: cancers of the breast, colon, larynx, lung, pancreas or prostate,
CYP1B1 gene combined genotype 4329 C/C, 142 C/C, 355 G/T being indicative of significantly increased predisposition to at least one of the following cancers: cancers of the breast or colon,
CYP1B1 gene combined genotype 4329 C/C, 142 G/C, 355 T/T being indicative of significantly increased predisposition to larynx cancer,
CYP1B1 gene combined genotype 4329 C/C, 142 G/G, 355 G/G being indicative of significantly increased predisposition to colon cancer,
CYP1B1 gene combined genotype 4329 G/C, 142 G/G, 355 G/T being indicative of significantly increased predisposition to prostate cancer,
CYP1B1 gene combined genotype 4329 G/C, 142 C/C, 355 G/G being indicative of significantly increased predisposition to breast cancer,
CYP1B1 gene combined genotype 4329 G/C, 142 C/C, 355 G/T being indicative of significantly increased predisposition to at least one of the following cancers: cancers of the breast or colon,
CYP1B1 gene combined genotype 4329 G/G, 142 C/C, 355 G/G being indicative of significantly increased predisposition to kidney cancer,
CYP1B1 gene combined genotype 4329 C/C, 355 T/T being indicative of significantly increased predisposition to at least one of the following cancers: cancers of the breast, larynx, pancreas or prostate,
CYP1B1 gene combined genotype 4329 C/C, 355 G/T being indicative of significantly increased predisposition to lung cancer,
CYP1B1 gene combined genotype 4329 G/G, 355 G/G being indicative of significantly increased predisposition to kidney cancer,
CYP1B1 gene combined genotype 4329 G/G, 355 T/T being indicative of significantly increased predisposition to colon cancer,
CYP1B1 gene combined genotype 4329 C/C, 142 G/G being indicative of significantly increased predisposition to at least one of the following cancers: cancers of the breast or prostate,
CYP1B1 gene combined genotype 4329 C/C, 142 G/C being indicative of significantly increased predisposition to larynx cancer,
CYP1B1 gene combined genotype 4329 G/C, 142 C/C being indicative of significantly increased predisposition to breast cancer,
CYP1B1 gene combined genotype 4329 G/G, 142 C/C being indicative of significantly increased predisposition to kidney cancer,
CYP1B1 gene combined genotype 142 C/C, 355 G/G being indicative of significantly increased predisposition to at least one of the following cancers: cancers of the breast, kidney or lung,
CYP1B1 gene combined genotype 142 C/C, 355 G/T being indicative of significantly increased predisposition to at least one of the following cancers: cancers of the breast or colon,
CYP1B1 gene combined genotype 142 C/C, 355 T/T being indicative of significantly increased predisposition to colon cancer,
CYP1B1 gene combined genotype 142 G/C, 355 T/T being indicative of significantly increased predisposition to larynx cancer,
CYP1B1 gene combined genotype 142 G/G, 355 G/G being indicative of significantly increased predisposition to colon cancer, or
CYP1B1 gene combined genotype 142 G/G, 355 T/T being indicative of significantly increased predisposition to at least one of the following cancers: cancers of the breast or prostate.

5. The method of claim 3, wherein the identified genotype of the CYP1B1 gene indicative of significantly decreased predisposition to cancer is the following combined genotype:
CYP1B1 gene combined genotype 4329 C/C, 142 G/G, 355 G/G being indicative of significantly decreased predisposition to breast cancer,
CYP1B1 gene combined genotype 4329 C/C, 142 G/C, 355 G/G being indicative of significantly decreased predisposition to at least one of the following cancers: cancers of the breast or prostate,
CYP1B1 gene combined genotype 4329 C/C, 142 G/G, 355 G/T being indicative of significantly decreased predisposition to breast cancer,
CYP1B1 gene combined genotype 4329 G/C, 142 G/C, 355 G/G being indicative of significantly decreased predisposition to at least one of the following cancers: breast, larynx, lung or prostate,
CYP1B1 gene combined genotype 4329 G/G, 142 C/C, 355 G/T being indicative of significantly decreased predisposition to breast cancer,
CYP1B1 gene combined genotype 4329 G/G, 142 G/C, 355 G/T being indicative of significantly decreased predisposition to at least one of the following cancers: cancers of the breast, lung or prostate,
CYP1B1 gene combined genotype 4329 G/G, 355 T/T being indicative of significantly decreased predisposition to breast cancer,
CYP1B1 gene combined genotype 4329 G/G, 355 G/T being indicative of significantly decreased predisposition to at least one of the following cancers: cancers of the breast, larynx, lung, pancreas or prostate,
CYP1B1 gene combined genotype 4329 G/C, 355 T/T being indicative of significantly decreased predisposition to breast cancer,
CYP1B1 gene combined genotype 4329 G/C, 142 G/G being indicative of significantly decreased predisposition to colon cancer,
CYP1B1 gene combined genotype 4329 G/G, 142 G/C being indicative of significantly decreased predisposition to at least one of the following cancers: cancers of the breast, larynx, lung or pancreas,
CYP1B1 gene combined. genotype 4329 G/C, 142 G/C being indicative of significantly decreased predisposition to at least one of the following cancers: cancers of the breast or prostate,
CYP1B1 gene combined genotype 142 C/C, 355 G/T being indicative of significantly decreased predisposition to lung cancer,
CYP1B1 gene combined genotype 142 G/C, 355 G/G being indicative of significantly decreased predisposition to at least one of the following cancers: cancers of the breast, larynx, lung or prostate,
CYP1B1 gene combined genotype 142 G/G, 355 G/G being indicative of significantly decreased predisposition to the prostate cancer, or
CYP1B1 gene combined genotype 142 G/G, 355 G/T being indicative of significantly decreased predisposition to at least one of the following cancers: cancers of the breast or larynx.

6. A method for detecting a predisposition to cancer in a human subject, comprising detecting in a biological sample from the subject a germline alteration in sequence of CYP1B1 gene and identification of the CYP1B1 genotype, wherein the genotype is indicative of predisposition to at least one of the following cancers: cancers of the breast, colon, kidney, larynx, lung, pancreas, prostate, thyroid and ovarian.

7. The method of claim 6, wherein the examined alteration in sequence of CYP1B1 gene is at least one of the following alteration:
variants of A119S alteration: 355T/T, 355G/T or 355G/G,
variants of R48G alteration: 142G/C, 142C/C, 142G/G
variants of L432V alteration: 4329C/C, 4329G/C, 4329G/G
or other CYP1B1 alterations with analogous properties.

8. The method of claim 6, wherein the identified genotype of CYP1B1 gene being indicative of significantly increased predisposition to cancer is at least one of the following combined genotype:
CYP1B1 gene combined genotype 4329 C/C, 142 G/G, 355 T/T being indicative of significantly increased predisposition to at least one of the following cancers: cancers of the breast, colon, larynx, lung, pancreas or prostate,
CYP1B1 gene combined genotype 4329 C/C, 142 C/C, 355 G/T being indicative of significantly increased predisposition to at least one of the following cancers: cancers of the breast or colon,
CYP1B1 gene combined genotype 4329 C/C, 142 G/C, 355 T/T being indicative of significantly increased predisposition to larynx cancer,
CYP1B1 gene combined genotype 4329 C/C, 142 G/G, 355 G/G being indicative of significantly increased predisposition to colon cancer,
CYP1B1 gene combined genotype 4329 G/C, 142 G/G, 355 G/T being indicative of significantly increased predisposition to prostate cancer,
CYP1B1 gene combined genotype 4329 G/C, 142 C/C, 355 G/G being indicative of significantly increased predisposition to breast cancer,
CYP1B1 gene combined genotype 4329 G/C, 142 C/C, 355 G/T being indicative of significantly increased predisposition to at least one of the following cancers: cancers of the breast or colon,
CYP1B1 gene combined genotype 4329 G/G, 142 C/C, 355 G/G being indicative of significantly increased predisposition to kidney cancer,
CYP1B1 gene combined genotype 4329 C/C, 355 T/T being indicative of significantly increased predisposition to at least one of the following cancers: cancers of the breast, larynx, pancreas or prostate,
CYP1B1 gene combined genotype 4329 C/C, 355 G/T being indicative of significantly increased predisposition to lung cancer,
CYP1B1 gene combined genotype 4329 G/G, 355 G/G being indicative of significantly increased predisposition to kidney cancer,
CYP1B1 gene combined genotype 4329 G/G, 355 T/T being indicative of significantly increased predisposition to colon cancer,
CYP1B1 gene combined genotype 4329 C/C, 142 G/G being indicative of significantly increased predisposition to at least one of the following cancers: cancers of the breast or prostate,
CYP1B1 gene combined genotype 4329 C/C, 142 G/C being indicative of significantly increased predisposition to larynx cancer,
CYP1B1 gene combined genotype 4329 G/C, 142 C/C being indicative of significantly increased predisposition to breast cancer,
CYP1B1 gene combined genotype 4329 G/G, 142 C/C being indicative of significantly increased predisposition to kidney cancer,
CYP1B1 gene combined genotype 142 C/C, 355 G/G being indicative of significantly increased predisposition to at least one of the following cancers: cancers of the breast, kidney or lung,
CYP1B1 gene combined genotype 142 C/C, 355 G/T being indicative of significantly increased predisposition to at least one of the following cancers: cancers of the breast or colon,
CYP1B1 gene combined genotype 142 C/C, 355 T/T being indicative of significantly increased predisposition to colon cancer,
CYP1B1 gene combined genotype 142 G/C, 355 T/T being indicative of significantly increased predisposition to larynx cancer,
CYP1B1 gene combined genotype. 142 G/G, 355 G/G being indicative of significantly increased predisposition to colon cancer,
CYP1B1 gene combined genotype 142 G/G, 355 T/T being indicative of significantly increased predisposition to at least one of the following cancers: cancers of the breast or prostate.

9. The method of claim 6, wherein the identified genotype of CYP1B1 gene being indicative of significantly decreased predisposition to cancer is at least one of the following combined genotype:
CYP1B1 gene combined genotype 4329 C/C, 142 G/G, 355 G/G being indicative of significantly decreased predisposition to breast cancer,
CYP1B1 gene combined genotype 4329 C/C, 142 G/C, 355 G/G being indicative of significantly decreased predisposition to at least one of the following cancers: cancers of the breast or prostate,
CYP1B1 gene combined genotype 4329 C/C, 142 G/G, 355 G/T being indicative of significantly decreased predisposition to breast cancer,
CYP1B1 gene combined genotype 4329 G/C, 142 G/C, 355 G/G being indicative of significantly decreased predisposition to at least one of the following cancers: breast, larynx, lung or prostate,
CYP1B1 gene combined genotype 4329 G/G, 142 C/C, 355 G/T being indicative of significantly decreased predisposition to breast cancer,
CYP1B1 gene combined genotype 4329 G/G, 142 G/C, 355 G/T being indicative of significantly decreased predisposition to at least one of the following cancers: cancers of the breast, lung or prostate,
CYP1B1 gene combined genotype 4329 G/G, 355 T/T being indicative of significantly decreased predisposition to breast cancer,
CYP1B1 gene combined genotype 4329 G/G, 355 G/T being indicative of significantly decreased predisposition to at least one of the following cancers: cancers of the breast, larynx, lung, pancreas or prostate,
CYP1B1 gene combined genotype 4329 G/C, 355 T/T being indicative of significantly decreased predisposition to breast cancer,
CYP1B1 gene combined genotype 4329 G/C, 142 G/G being indicative of significantly decreased predisposition to colon cancer,
CYP1B1 gene combined genotype 4329 G/G, 142 G/C being indicative of significantly decreased predisposition to at least one of the following cancers: cancers of the breast, larynx, lung or pancreas,
CYP1B1 gene combined genotype 4329 G/C, 142 G/C being indicative of significantly decreased predisposition to at least one of the following cancers: cancers of the breast or prostate,
CYP1B1 gene combined genotype 142 C/C, 355 G/T being indicative of significantly decreased predisposition to lung cancer,
CYP1B1 gene combined genotype 142 G/C, 355 G/G being indicative of significantly decreased predisposition to at least one of the following cancers: cancers of the breast, larynx, lung or prostate,
CYP1B1 gene combined genotype 142 G/G, 355 G/G being indicative of significantly decreased predisposition to the prostate cancer,
CYP1B1 gene combined genotype 142 G/G, 355 G/T being indicative of significantly decreased predisposition to at least one of the following cancers: cancers of the breast or larynx.

10. The method of claim 6, wherein genetic testing is performed among adults.

11. The method of claim 6, wherein presence of germline alteration is detected by analysis of DNA, RNA or proteins.

12. The method according to claim 11, wherein DNA or RNA testing is performed with the use of any technique of indirect mutation detection, selected among ASA-, ASO-, RFLP-PCR, microarrays or methods of direct mutation detection such as sequencing.

13. The method according to claim 11, wherein the presence of the polypeptide encoded by CYP1B1 allele with germline alteration is detected with the use of antibodies or other substances specific for this polypeptide or its fragment.

14. The method of claim 6, wherein the human subject is a person of Polish ethnic origin.

15. Diagnostic composition for detection predisposition to cancer in human subject, comprising at least two different oligonucleotides allowing amplification of region of genome of said human subject containing at least one of the following alteration:
variants of A119S alteration: 355T/T, 355G/T or 355G/G,
variants of R48G alteration: 142G/C, 142C/C, 142G/G
variants of L432V alteration: 4329C/C, 4329G/C, 4329G/G
or other alterations in linkage disequilibrium with above variants, wherein the detected predisposition to cancer is significantly increased/decreased predisposition to at least one of the following cancers: cancers of the breast, colon, kidney, larynx, lung, pancreas, prostate, thyroid and ovarian.

16. Diagnostic composition according to claim 15, characterized in that the amplified region is used for identification of the CYP1B1 genotype, wherein specific identified combined CYP1B1 genotypes are indicative of significantly increased predisposition to specific cancer, and specific identified combined CYP1B1 genotypes are indicative of significantly decreased predisposition to cancer.

17.-20. (canceled)
